(19) United States
(12) Patent Application Publication
(10) Pub. No.: US 2023/0287508 A1
(43) Pub. Date: Sep. 14, 2023

(54) METHODS AND COMPOSITIONS FOR DETECTING STRUCTURAL REARRANGEMENTS IN A GENOME

(71) Applicant: Roche Sequencing Solutions, Inc., Pleasanton, CA (US)

(72) Inventors: Alexander Lovejoy, Pleasanton, CA (US); Melissa Loyzer, Pleasanton, CA (US)

(21) Appl. No.: 18/000,699

(22) PCT Filed: Jun. 1, 2021
(86) PCT No.: PCT/EP2021/064652

§ 371 Date: Dec. 5, 2022

Related U.S. Application Data
(60) Provisional application No. 63/036,064, filed on Jun. 8, 2020.

(51) Int. Cl. C12Q 1/6886; C12N 15/10

(52) U.S. Cl. CPC C12Q 1/6886; C12N 15/1065; C12Q 2600/156

(57) ABSTRACT

Disclosed are compositions, kits, and methods for detecting gene fusions involving an unknown fusion partner using locked nucleic acid primers. In some embodiments, the compositions include a compound including at least two nucleotide sequences which are joined, directly or indirectly, through a 5′ to 5′ linkage. In some embodiments, the compound further includes a spacer moiety and/or a cleavage moiety.

### FIELD OF THE DISCLOSURE

The present disclosure relates to the field of genomics. More specifically, the present disclosure relates to the field of detecting genomic rearrangements.

### BACKGROUND OF THE DISCLOSURE

Gene fusions are a common occurrence in cancer. Some gene fusions are cancer driver mutations for which targeted therapies have been developed. The ability to detect gene fusions can be helpful in detecting and diagnosing cancer, in tracking tumor burden over time, and for identifying the best individualized treatment for a cancer patient. Traditional methods of detecting genomic rearrangements involve cumbersome multi-step procedures such as haplotype fusion PCR and ligation haplotyping, see Turner et al., (2008) Long range, high throughput haplotype determination via haplotype fusion PCR and ligation haplotyping, Nucl. Acids Res. 36:e82. More recent next-generation sequencing-based techniques are able to identify a variety of gene fusions. However, this requires a large amount of sequencing to capture and validate a sufficient number of fusion sequences. The cost and complexity of this approach makes it ill-suited for clinical use.

For some genes, detecting gene fusions is further complicated by the occurrence of multiple fusion partners. For example, neurotrophic tropomyosin receptor kinase genes (NTRK 1, 2 and 3) may fuse with any number of N-terminal (5′-) partners, see Solomon et al. (2019) Identifying patients with NTRK fusion cancer, Ann. Oncol. November;30 Suppl 8:viii16-viii22. Because effective therapy for activated NTRK exists, a cost-effective clinical test for identifying qualified patients with NTRK gene fusions is critical. Similarly, fibroblast growth factor receptor genes (FGFR 2 and 3) may fuse with any number of C-terminal (3′-) partners resulting in constitutively active receptor-kinase protein, see Facchinetti et al. (2020) Facts and New Hopes on Selective FGFR Inhibitors in Solid Tumors, Clin. Cancer Res. 2020 Feb. 15;26(4):764-774. With multiple FGFR kinase inhibitors in development, a practical clinical test for identifying qualifying patients with FGFR gene fusions in various tumor types is needed.

### SUMMARY OF THE DISCLOSURE

Based on the foregoing, there is a need to identify gene fusions with less sequencing at a lower cost in order to increase patient access to potentially lifesaving therapies

The present disclosure is directed to compositions, kits, and methods for detecting one or more gene fusions in a nucleic acid sample. In some embodiments, the present disclosure provides one or more compounds each having Formula (I):

[Olig1]—([R¹]ₒ—[R²]ₚ)_{q}—[L¹]ₜ—[Z]—[L²]ᵤ—[W]ᵥ—[Olig2] (I),

wherein
o is 0 or 1;
p is 0 or 1;
q is 0 or 1;
t is 0, 1 or 2;
u is 0, 1 or 2;
v is 0 or 1;
R¹ is an oligonucleotide having between about 1 and about 24 nucleotides;
R² is a substituted or unsubstituted, saturated or unsaturated, linear or cyclic aliphatic group having between 2 and about 48 carbon atoms, optionally substituted with one or more heteroatoms selected from O, N, or S;
L¹ and L² are independently a substituted or unsubstituted, saturated or unsaturated, linear or cyclic aliphatic group having between 1 and about 16 carbon atoms, optionally including with one or more heteroatoms selected from O, N, or S, and optionally including one or more carbonyl groups;
Z is a moiety selected from a triazole, a dihydropyridazine, a phosphate linkage, an amide linkage, a thioether linkage, an isooxazoline, a hydrozone, an oxime ether, and a chloro-s-triazine linkage;
W is a substituted or unsubstituted, saturated or unsaturated, aliphatic or aromatic group having between 1 and about 12 carbon atoms, optionally substituted with one or more heteroatoms selected from O, N, S, provided that W includes at least one photocleavable, enzymatically cleavable, chemically cleavable, or pH sensitive group;
Olig1 is an oligonucleotide comprising between about 1 and about 30 nucleotides; and
Olig2 is an oligonucleotide comprising between about 1 and about 30 nucleotides.

In some embodiments, the compounds of Formula (I) may be used to facilitate the detection of gene fusions. In that regard, the present disclosure is also directed to methods of detecting gene fusions using one or more of the compounds of Formula (I). In some embodiments, the compounds of Formula (I) facilitate the capture of a gene fusion where one fusion partner is utilized in the detection, amplification, and/or sequencing of one or more gene fusions in a sample (e.g. a histological sample, a cytological sample, etc.). These and other aspects of the present disclosure are further described herein.

In a first aspect of the present disclosure is a method of detecting a gene fusion in a nucleic acid sample, the method comprising: (a) contacting a sample with a polymerase (e.g. a nucleic acid polymerase having a polymerase activity and a strand displacement activity), and with a compound having Formula (I):

[Olig1]—([R¹]ₒ—[R²]ₚ)_{q}—[L¹]ₜ—[Z]—[L²]ᵤ—[W]ᵥ—[Olig2] (I),

wherein
o is 0 or 1;
p is 0 or 1;
q is 0 or 1;
t is 0, 1 or 2;
u is 0, 1 or 2;
v is 0 or 1;
R¹ is an oligonucleotide having between about 1 and about 24 nucleotides;
R² is a substituted or unsubstituted, saturated or unsaturated, linear or cyclic aliphatic group having between 2 and about 48 carbon atoms, optionally substituted with one or more heteroatoms selected from O, N, or S;
L¹ and L² are independently a substituted or unsubstituted, saturated or unsaturated, linear or cyclic aliphatic group having between 1 and about 16 carbon atoms, optionally including with one or more heteroatoms selected from O, N, or S, and optionally including one or more carbonyl groups;
Z is a moiety selected from a triazole, a dihydropyridazine, a phosphate linkage, an amide linkage, a thioether linkage, an isooxazoline, a hydrozone, an oxime ether, and a chloro-s-triazine linkage;
W is a substituted or unsubstituted, saturated or unsaturated, aliphatic or aromatic group having between 1 and about 12 carbon atoms, optionally substituted with one or more heteroatoms selected from O, N, S, provided that W includes at least one photocleavable, enzymatically cleavable, chemically cleavable, or pH sensitive group;
Olig1 is an oligonucleotide comprising between about 1 and about 30 nucleotides and comprising an anchor sequence capable of hybridizing to a known fusion partner, and wherein Olig1 has a non-extendable 3′ end; and
Olig2 is an oligonucleotide comprising between about 1 and about 30 nucleotides and comprising an extendable 3′ end; and
(b) extending the 3′-end of Olig2 of the compound having Formula (I) with the polymerase, thereby producing an extension product. In some embodiments, the extension product comprises a copy of a portion of an unknown fusion partner, a portion of the known fusion partner, and a fusion breakpoint, thereby forming a first strand copy of a gene fusion.

In some embodiments, Olig2 comprises a random sequence. In some embodiments, the random sequence comprises between 2 and 20 nucleotides.

In some embodiments, o+p=1, and q is 1. In some embodiments, R² comprises a moiety having the structure of Formula (IVB):

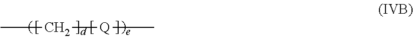

wherein d and e are integers each independently ranging from 1 to 32; Q is a bond, O, S, or N(R_{c})(R_{d}); and R_{c} and R_{d} are independently CH₃ or H. In some embodiments, R² includes a moiety having the structure of Formula (IVC):

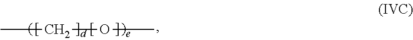

wherein d and e are integers each independently ranging from 1 to 32. In some embodiments, d and e range from 1 to 16. In some embodiments, d an e range from 2 to 8. In some embodiments, the method further comprises forming a second strand copy of the gene fusion by copying the first strand copy, thereby forming a double-stranded copy of the gene fusion.

In some embodiments, R¹ comprises between about 2 and about 9 nucleotides. In some embodiments, R¹ includes between 4 and 8 nucleotides.

In some embodiments, v is 1. In some embodiments, the method further comprises cleaving the photocleavable, enzymatically cleavable, chemically cleavable, or pH sensitive group of the linked primer.

In some embodiments, v is 0 and Olig2 comprises a cleavage site including a uracil-containing nucleotide.

In some embodiments, at least one of L¹ or L² comprises a substituted or unsubstituted, saturated or unsaturated, linear or cyclic aliphatic group having between 1 and about 4 carbon atoms, optionally substituted with one or more heteroatoms selected from O, N, or S, and optionally including one or more carbonyl groups. In some embodiments, the aliphatic group is linear. In some embodiments, the aliphatic group is linear and unsubstituted. In some embodiments, the aliphatic group is linear and unsubstituted and includes one carbonyl group. In some embodiments, the aliphatic group is linear and substituted.

In some embodiments, the method further comprises sequencing the copy of the gene fusion. In some embodiments, the method further comprises forming a library of double-stranded copies of the gene fusions. In some embodiments, the forming the library comprises: attaching adaptors to copies of gene fusions wherein adaptors comprise barcodes and primer binding sites. In some embodiments, the method further comprises amplifying at least a portion of the formed library via universal amplification. In some embodiments, the method further comprises sequencing at least a portion of the formed library. In some embodiments, the barcodes comprise unique molecular barcodes (UID) and sequencing comprises grouping the sequence of library nucleic acids by UID into families, determining consensus read for each family, and aligning the consensus read to the reference genome thereby determining the sequence of the gene fusion.

In some embodiments, the method further comprises amplifying the copy strand by a method comprising: (a) partitioning the sample comprising the copy strand into a plurality of reaction volumes; wherein each reaction volume comprises a forward and reverse amplification primers capable of hybridizing to the copy strand and the complement of the copy strand, and a first detectably-labeled probe; (b) performing an amplification reaction, wherein the reaction comprises a step of detection with the probe; (c) determining a number of reaction volumes where the probe has been detected thereby detecting the gene fusion. In some embodiments, the reaction volumes are droplets. In some embodiments, the detectable label comprises a combination of a fluorophore and a quencher.

In some embodiments, multiple fusions are detected in the sample by contacting the sample with two or more of the compounds having Formula (I). In some embodiments, Olig1 of each of the two or more compounds of Formula (I) are capable of hybridizing to a gene selected from ALK, PPARG, BRAF, EGFR, FGFR1, FGFR2, FGFR3, MET, NRG1, NTRK1, NTRK2, NTRK3, RET, ROS1, AXL, PDGFRA, PDGFB , ABL1, ABL2, AKT1, AKT2, AKT3, ARHGAP26, BRD3, BRD4, CRLF2, CSF1R, EPOR, ERBB2, ERBB4, ERG, ESR1, ESRRA, ETV1, ETV4, ETV5, ETV6, EWSR1, FGR, IL2RB, INSR, JAK1, JAK2, JAK3, KIT, MAML2, MAST1, MAST2, MSMB, MUSK, MYB, MYC, NOTCH1, NOTCH2, NUMBL, NUT, PDGFRB, PIK3CA, PKN1, PRKCA, PRKCB, PTK2B, RAF1, RARA, RELA, RSPO2, RSPO3, SYK, TERT, TFE3, TFEB, THADA, TMPRSS2, TSLP, TY, BCL2, BCL6, BCR, CAMTA1, CBFB, CCNB3, CCND1, CIC, CRFL2, DUSP22, EPC1, FOXO1, FUS, GLI1, GLIS2, HMGA2, JAZF1, KMT2A, MALT1, MEAF6, MECOM, MKL1, MKL2, MTB, NCOA2, NUP214, NUP98, PAX5, PDGFB, PICALM, PLAG1, RBM15, RUNX1, RUNX1T1, SS18, STAT6, TAF15, TAL1, TCF12, TCF3, TFG, TYK2, USP6, YWHAE, AR, BRCA1, BRCA2, CDKN2A, ERB84, FLT3, KRAS, MDM4, MYBL1, NF1, NOTCH4, NUTM1, PRKACA, PRKACB, PTEN, RAD51B, and RB1.

In a second aspect of the present disclosure is a compound having Formula (I),

[Olig1]—([R¹]ₒ—[R²]ₚ)_{q}—[L¹]ₜ—[Z]—[L²]ᵤ—[W]ᵥ—[Olig2] (I),

wherein
o is 0 or 1;
p is 0 or 1;
q is 0 or 1;
t is 0, 1 or 2;
u is 0, 1 or 2;
v is 0 or 1;
R¹ is an oligonucleotide having between about 1 and about 24 nucleotides;
R² is a substituted or unsubstituted, saturated or unsaturated, linear or cyclic aliphatic group having between 2 and about 48 carbon atoms, optionally substituted with one or more heteroatoms selected from O, N, or S;
L¹ and L² are independently a substituted or unsubstituted, saturated or unsaturated, linear or cyclic aliphatic group having between 1 and about 16 carbon atoms, optionally including with one or more heteroatoms selected from O, N, or S, and optionally including one or more carbonyl groups;
Z is a moiety selected from a triazole, a dihydropyridazine, a phosphate linkage, an amide linkage, a thioether linkage, an isooxazoline, a hydrozone, an oxime ether, and a chloro-s-triazine linkage;
W is a substituted or unsubstituted, saturated or unsaturated, aliphatic or aromatic group having between 1 and about 12 carbon atoms, optionally substituted with one or more heteroatoms selected from O, N, S, provided that W includes at least one photocleavable, enzymatically cleavable, chemically cleavable, or pH sensitive group;
Olig1 is an oligonucleotide having between about 1 and about 30 nucleotides, and wherein Olig1 has a non-extendable 3′ end; and
Olig2 is an oligonucleotide having between about 1 and about 30 nucleotides, and wherein Olig2 has an extendable 3′ end.

In some embodiments, R² comprises a substituted or unsubstituted, saturated or unsaturated, linear or cyclic aliphatic group having between 2 and about 32 carbon atoms, optionally substituted with one or more heteroatoms selected from O, N, or S, and optionally including one or more carbonyl groups. In some embodiments, R² comprises a moiety having the structure of Formula (IVA):

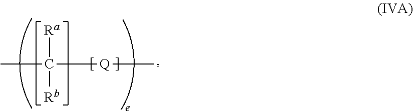

wherein d and e are integers each independently ranging from 1 to 32; Q is a bond, O, S, N(R^{c})(R^{d}) or a quaternary amine (N+H(R^{c})(R^{d})); R^{a} and R^{b} are independently H, a C₁-C₄ alkyl group, F, Cl, or N(R^{c})(R^{d}); and R^{c} and R^{d} are independently CH₃ or H. In some embodiments, d is 2 or 3; and wherein e is an integer ranging from between 1 and 12. In some embodiments, R² comprises a moiety having the structure of Formula (IVB):

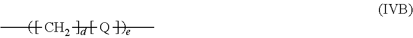

wherein d and e are integers each independently ranging from 1 to 32; Q is a bond, O, S, or N(R_{c})(R_{d}); and R_{c} and R_{d} are independently CH₃ or H. In some embodiments, d is 2 or 3; and wherein e is an integer ranging from between 1 and 12. In some embodiments, d is 2 or 3; and wherein e is an integer ranging from between 1 and 8.

In some embodiments, at least one of L¹ or L² comprises a substituted or unsubstituted, saturated or unsaturated, linear or cyclic aliphatic group having between 1 and about 4 carbon atoms, optionally substituted with one or more heteroatoms selected from O, N, or S, and optionally including one or more carbonyl groups.

In some embodiments, o+p=1, and q is 1. In some embodiments, R¹ comprises between about 1 and about 16 nucleotides. In some embodiments, R¹ comprises between about 2 and about 9 nucleotides. In some embodiments, R² includes a moiety having the structure of Formula (IVC):

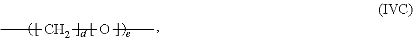

wherein d and e are integers each independently ranging from 1 to 32. In some embodiments, d is 2 or 3; and wherein e is an integer ranging from between 1 and 12. In some embodiments, d is 2 or 3; and wherein e is an integer ranging from between 1 and 8. In some embodiments, d is 2; and wherein e is an integer ranging from between 1 and 12. In some embodiments, d is 2; and wherein e is an integer ranging from between 1 and 8. In some embodiments, d is 2; and wherein e is an integer ranging from between 2 and 6.

In some embodiments, o is 0 and p and q are both 1, R¹ comprises at least one PEG group, and L¹ comprises at least one carbonyl moiety. In some embodiments, o is 0 and p and q are both 1, R¹ comprises at least two PEG groups, and L¹ comprises at least one carbonyl moiety. In some embodiments, o is 0 and p and q are both 1, R¹ comprises at least three PEG groups, and L¹ comprises at least one carbonyl moiety. In some embodiments, o is 0 and p and q are both 1, R¹ comprises at least four PEG groups, and L¹ comprises at least one carbonyl moiety. In some embodiments, o is 0 and p and q are both 1, R¹ comprises at least six PEG groups, and L¹ comprises at least one carbonyl moiety. In some embodiments, o is 0 and p and q are both 1, R¹ comprises at least eight PEG groups, and L¹ comprises at least one carbonyl moiety. In some embodiments, o is 0 and p and q are both 1, R¹ comprises at least twelve PEG group, and L¹ comprises at least one carbonyl moiety.

In some embodiments, Olig2 comprises a barcode. In some embodiments, the barcode is one or more of unique molecular barcode (UID), a sample barcode, and an identifying tag. In some embodiments, Olig2 comprises a universal primer binding site. In some embodiments, v is 0 and Olig2 includes a cleavage site including a uracil-containing nucleotide. In some embodiments, Olig2 comprises a random nucleotide sequence.

In some embodiments, at least a portion of Olig1 comprises a nucleotide sequence is capable of hybridizing to a gene selected from the group consisting of ALK, PPARG, BRAF, EGFR, FGFR1, FGFR2, FGFR3, MET, NRG1, NTRK1, NTRK2, NTRK3, RET, ROS1, AXL, PDGFRA, PDGFB , ABL1, ABL2, AKT1, AKT2, AKT3, ARHGAP26, BRD3, BRD4, CRLF2, CSF1R, EPOR, ERBB2, ERBB4, ERG, ESR1, ESRRA, ETV1, ETV4, ETV5, ETV6, EWSR1, FGR, IL2RB, INSR, JAK1, JAK2, JAK3, KIT, MAML2, MAST1, MAST2, MSMB, MUSK, MYB, MYC, NOTCH1, NOTCH2, NUMBL, NUT, PDGFRB, PIK3CA, PKN1, PRKCA, PRKCB, PTK2B, RAF1, RARA, RELA, RSPO2, RSPO3, SYK, TERT, TFE3, TFEB, THADA, TMPRSS2, TSLP, TY, BCL2, BCL6, BCR, CAMTA1, CBFB, CCNB3, CCND1, CIC, CRFL2, DUSP22, EPC1, FOXO1, FUS, GLI1, GLIS2, HMGA2, JAZF1, KMT2A, MALT1, MEAF6, MECOM, MKL1, MKL2, MTB, NCOA2, NUP214, NUP98, PAX5, PDGFB, PICALM, PLAG1, RBM15, RUNX1, RUNX1T1, SS18, STAT6, TAF15, TAL1, TCF12, TCF3, TFG, TYK2, USP6, YWHAE, AR, BRCA1, BRCA2, CDKN2A, ERB84, FLT3, KRAS, MDM4, MYBL1, NF1, NOTCH4, NUTM1, PRKACA, PRKACB, PTEN, RAD51B, and RB1.

In some embodiments, Olig1 is not extendable. In some embodiments, Olig2 is extendable. In some embodiments, Olig1 comprises between 1 and about 10 nucleotides. In some embodiments, Olig2 comprises between 1 and about 10 nucleotides.

In some embodiments, a size of the group —([R¹]ₒ—[R²]ₚ)_{q}— ranges from between about 15 Angstroms to about 400 Angstroms. In some embodiments, a size of the group —([R¹]ₒ—[R²]ₚ)_{q}— ranges from between about 15 Angstroms to about 200 Angstroms. In some embodiments, a size of the group —([R¹]ₒ—[R²]ₚ)_{q}— ranges from between about 15 Angstroms to about 100 Angstroms. In some embodiments, a size of the group —([R¹]ₒ13 [R²]ₚ)_{q}— ranges from between about 15 Angstroms to about 50 Angstroms. In some embodiments, a size of the group —([R¹]ₒ—[R²]ₚ)_{q}— ranges from between about 20 Angstroms to about 45 Angstroms. In some embodiments, a size of the group —([R¹]ₒ—[R²]ₚ)_{q}— ranges from between about 20 Angstroms to about 40 Angstroms.

In a third aspect of the present disclosure is a kit for detecting gene fusions, such as for detecting gene fusions between a known fusion partner and an unknown fusion partner, wherein the kit comprises (a) a DNA polymerase; and (b) a compound having Formula (I),

[Olig1]—([R¹]ₒ—[R²]ₚ)_{q}—[L¹]ₜ—[Z]—[L²]ᵤ—[W]ᵥ—[Olig2] (I),

wherein
o is 0 or 1;
p is 0 or 1;
q is 0 or 1;
t is 0, 1 or 2;
u is 0, 1 or 2;
vis 0 or 1;
R¹ is an oligonucleotide having between about 1 and about 24 nucleotides;
R² is a substituted or unsubstituted, saturated or unsaturated, linear or cyclic aliphatic group having between 2 and about 48 carbon atoms, optionally substituted with one or more heteroatoms selected from O, N, or S;
L¹ and L² are independently a substituted or unsubstituted, saturated or unsaturated, linear or cyclic aliphatic group having between 1 and about 16 carbon atoms, optionally including with one or more heteroatoms selected from O, N, or S, and optionally including one or more carbonyl groups;
Z is a moiety selected from a triazole, a dihydropyridazine, a phosphate linkage, an amide linkage, a thioether linkage, an isooxazoline, a hydrozone, an oxime ether, and a chloro-s-triazine linkage;
W is a substituted or unsubstituted, saturated or unsaturated, aliphatic or aromatic group having between 1 and about 12 carbon atoms, optionally substituted with one or more heteroatoms selected from O, N, S, provided that W includes at least one photocleavable, enzymatically cleavable, chemically cleavable, or pH sensitive group;
Olig1 is an oligonucleotide having between about 1 and about 30 nucleotides, and wherein Olig1 has a non-extendable 3′ end; and
Olig2 is an oligonucleotide having between about 1 and about 30 nucleotides, and wherein Olig2 has an extendable 3′ end.

In some embodiments, the kit further comprises a forward amplification primer and a reverse amplification primer. In some embodiments, Olig2 comprises at least one uracil-containing nucleotide, and wherein the kit further comprises a uracil-N-DNA glycosylase (UNG). In some embodiments, the DNA polymerase is a reverse transcriptase and the kit further comprises a thermostable DNA-dependent DNA polymerase.

In some embodiments, at least a portion of Olig1 comprises a nucleotide sequence is capable of hybridizing to a gene selected from the group consisting of ALK, PPARG, BRAF, EGFR, FGFR^{1,} FGFR^{2,} FGFR^{3,} MET, NRG1, NTRK1, NTRK2, NTRK3, RET, ROS1, AXL, PDGFRA, PDGFB , ABL1, ABL2, AKT1, AKT2, AKT3, ARHGAP26, BRD3, BRD4, CRLF2, CSF1R, EPOR, ERBB2, ERBB4, ERG, ESR1, ESRRA, ETV1, ETV4, ETV5, ETV6, EWSR1, FGR, IL2RB, INSR, JAK1, JAK2, JAK3, KIT, MAML2, MAST1, MAST2, MSMB, MUSK, MYB, MYC, NOTCH1, NOTCH2, NUMBL, NUT, PDGFRB, PIK3CA, PKN1, PRKCA, PRKCB, PTK2B, RAF1, RARA, RELA, RSPO2, RSPO3, SYK, TERT, TFE3, TFEB, THADA, TMPRSS2, TSLP, TY, BCL2, BCL6, BCR, CAMTA1, CBFB, CCNB3, CCND1, CIC, CRFL2, DUSP22, EPC1, FOXO1, FUS, GLI1, GLIS2, HMGA2, JAZF1, KMT2A, MALT1, MEAF6, MECOM, MKL1, MKL2, MTB, NCOA2, NUP214, NUP98, PAX5, PDGFB, PICALM, PLAG1, RBM15, RUNX1, RUNX1T1, SS18, STAT6, TAF15, TAL1, TCF12, TCF3, TFG, TYK2, USP6, YWHAE, AR, BRCA1, BRCA2, CDKN2A, ERB84, FLT3, KRAS, MDM4, MYBL1, NF1, NOTCH4, NUTM1, PRKACA, PRKACB, PTEN, RAD51B, and RB1.

In a fourth aspect of the present disclosure is a reaction vessel comprising a compound having Formula (I),

[Olig1]—([R¹]ₒ—[R²]ₚ)_{q}—[L¹]ₜ—[Z]—[L²]ᵤ—[W]ᵥ—[Olig2] (I),

wherein
o is 0 or 1;
p is 0 or 1;
q is 0 or 1;
t is 0, 1 or 2;
u is 0, 1 or 2;
v is 0 or 1;
R¹ is an oligonucleotide having between about 1 and about 24 nucleotides;
R² is a substituted or unsubstituted, saturated or unsaturated, linear or cyclic aliphatic group having between 2 and about 48 carbon atoms, optionally substituted with one or more heteroatoms selected from O, N, or S;
L¹ and L² are independently a substituted or unsubstituted, saturated or unsaturated, linear or cyclic aliphatic group having between 1 and about 16 carbon atoms, optionally including with one or more heteroatoms selected from O, N, or S, and optionally including one or more carbonyl groups;
Z is a moiety selected from a triazole, a dihydropyridazine, a phosphate linkage, an amide linkage, a thioether linkage, an isooxazoline, a hydrozone, an oxime ether, and a chloro-s-triazine linkage;
W is a substituted or unsubstituted, saturated or unsaturated, aliphatic or aromatic group having between 1 and about 12 carbon atoms, optionally substituted with one or more heteroatoms selected from O, N, S, provided that W includes at least one photocleavable, enzymatically cleavable, chemically cleavable, or pH sensitive group;
Olig1 is an oligonucleotide having between about 1 and about 30 nucleotides, and wherein Olig1 has a non-extendable 3′ end; and
Olig2 is an oligonucleotide having between about 1 and about 30 nucleotides, and wherein Olig2 has an extendable 3′ end.

In some embodiments, the reaction vessel includes at least one polymerase. In some embodiments, the at least one polymerase is a DNA polymerase. In some embodiments, the reaction vessel further comprises at least one buffer. In some embodiments, the reaction vessel further comprises at least one cofactor. In some embodiments, the reaction vessel further comprises dNTPs.

In a fifth aspect of the present disclosure is: (a) a compound having Formula (II):

[Oligo1]—([R¹]ₒ—[R²]ₚ)_{q}—[L¹]ₜ—[X] (I),

wherein
o is 0 or 1;
p is 0 or 1;
q is 1 or 2;
t is 0, 1 or 2;
R¹ is an oligonucleotide having between 1 and about 24 nucleotides;

R² is a substituted or unsubstituted, saturated or unsaturated, linear or cyclic aliphatic group having between 2 and about 48 carbon atoms, optionally substituted with one or more heteroatoms selected from O, N, or S;
L¹ is a substituted or unsubstituted, saturated or unsaturated, linear or cyclic aliphatic group having between 1 and about 16 carbon atoms, optionally substituted with one or more heteroatoms selected from O, N, or S, and optionally including one or more carbonyl groups;
X is a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, an amino group, or a phosphoramidite; and
Olig1 is an oligonucleotide having between about 1 to about 30 nucleotides; and
(b) a compound having Formula (III):

[Y]—[L²]ᵤ—[W]ᵥ—[Olig2] (III),

wherein
u is 0, 1 or 2;
v is 0 or 1;
Y is a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, an amino group, or a phosphoramidite;
L² is a substituted or unsubstituted, saturated or unsaturated, linear or cyclic aliphatic group having between 1 and 16 carbon atoms, optionally including with one or more heteroatoms selected from O, N, or S, and optionally including one or more carbonyl groups;
W is a substituted or unsubstituted, saturated or unsaturated, aliphatic or aromatic group having between 1 and 12 carbon atoms, optionally substituted with one or more heteroatoms selected from O, N, S, provided that W includes at least one photocleavable, enzymatically cleavable, chemically cleavable, or pH sensitive group; and
Olig2 is an oligonucleotide having between about 1 and about 30 nucleotides.

In some embodiments, Olig1 comprises anon-extendable 3′ end; and wherein Olig2 comprises an extendable 3′ end. In some embodiments, Olig1 comprises between 1 and about 10 nucleotides. In some embodiments, Olig2 comprises between 1 and about 10 nucleotides. In some embodiments, at least a portion of Olig1 is capable of hybridizing to a gene selected from the group consisting of ALK, PPARG, BRAF, EGFR, FGFR^{1,} FGFR^{2,} FGFR^{3,} MET, NRG1, NTRK1, NTRK2, NTRK3, RET, ROS1, AXL, PDGFRA, PDGFB , ABL1, ABL2, AKT1, AKT2, AKT3, ARHGAP26, BRD3, BRD4, CRLF2, CSF1R, EPOR, ERBB2, ERBB4, ERG, ESR1, ESRRA, ETV1, ETV4, ETV5, ETV6, EWSR1, FGR, IL2RB, INSR, JAK1, JAK2, JAK3, KIT, MAML2, MAST1, MAST2, MSMB, MUSK, MYB, MYC, NOTCH1, NOTCH2, NUMBL, NUT, PDGFRB, PIK3CA, PKN1, PRKCA, PRKCB, PTK2B, RAF1, RARA, RELA, RSPO2, RSPO3, SYK, TERT, TFE3, TFEB, THADA, TMPRSS2, TSLP, TY, BCL2, BCL6, BCR, CAMTA1, CBFB, CCNB3, CCND1, CIC, CRFL2, DUSP22, EPC1, FOXO1, FUS, GLI1, GLIS2, HMGA2, JAZF1, KMT2A, MALT1, MEAF6, MECOM, MKL1, MKL2, MTB, NCOA2, NUP214, NUP98, PAX5, PDGFB, PICALM, PLAG1, RBM15, RUNX1, RUNX1T1, SS18, STAT6, TAF15, TAL1, TCF12, TCF3, TFG, TYK2, USP6, YWHAE, AR, BRCA1, BRCA2, CDKN2A, ERB84, FLT3, KRAS, MDM4, MYBL1, NF1, NOTCH4, NUTM1, PRKACA, PRKACB, PTEN, RAD51B, and RB1.

In some embodiments, one of X or Y comprises an alkyne moiety; and the other of X or Y comprises an azide moiety. In some embodiments, the alkyne moiety is DBCO. In some embodiments, one of X or Y comprises a maleimide moiety; and the other of X or Y comprises a thiol moiety. In some embodiments, one of X or Y comprises a alkene moiety; and the other of X or Y comprises a tetrazine moiety. In some embodiments, comprise an amino moiety, and wherein the kit further comprises s-trichlorotriazine.

In some embodiments, R² comprises a substituted or unsubstituted, saturated or unsaturated, linear or cyclic aliphatic group having between 2 and about 32 carbon atoms, optionally substituted with one or more heteroatoms selected from O, N, or S, and optionally including one or more carbonyl groups. In some embodiments, R² comprises a moiety having the structure of Formula (IVA):

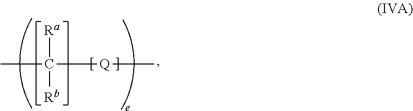

wherein d and e are integers each independently ranging from 1 to 32; Q is a bond, O, S, N(R^{c})(R^{d}) or a quaternary amine (N⁺H(R^{c})(R^{d})); R^{a} and R^{b} are independently H, a C₁-C₄ alkyl group, F, Cl, or N(^{Rc})(R^{d}); and R^{c} and R^{d} are independently CH₃ or H. In some embodiments, d is 2; and e is an integer ranging from 1 to about 12. In some embodiments, R² comprises a moiety having the structure of Formula (IVB):

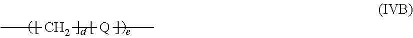

wherein d and e are integers each independently ranging from 1 to 32; Q is a bond, O, S, or N(R_{c})(R_{d}); and R_{c} and R_{d} are independently CH₃ or H. In some embodiments, is 2; and e is an integer ranging from 1 to about 12. In some embodiments, d is 2; and e is an integer ranging from 1 to about 6.

In some embodiments, o+p=1, and q is 1. In some embodiments, R¹ comprises between about 2 and about 9 nucleotides. In some embodiments, R² includes a moiety having the structure of Formula (IVC):

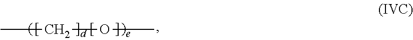

wherein d and e are integers each independently ranging from 1 to 32. In some embodiments, d is 2; and e is an integer ranging from 1 to about 12. In some embodiments, d is 2; and e is an integer ranging from 1 to about 6. In some embodiments, o is 0 and p and q are both 1, and L includes at least one PEG group.

In some embodiments, Olig2 comprises a barcode. In some embodiments, barcode is one or more of unique molecular barcode (UID), sample barcode, and an identifying tag. In some embodiments, Olig2 comprises a universal primer binding site. In some embodiments, v is 0 and Olig2 includes a cleavage site including a uracil-containing nucleotide.

In some embodiments, the kit further comprises a polymerase. In some embodiments, the polymerase is a DNA polymerase. In some embodiments, the kit further comprises a nucleic acid sample comprising at least one gene fusion. In some embodiments, the kit further comprises an aliquot of a master mix.

### BRIEF DESCRIPTION OF THE DRAWINGS

FIG. 1 is a diagram illustrating the steps of annealing and extending the compound of Formula (I).

FIG. 2 is a diagram illustrating the steps of strand displacement and strand cleavage liberating a copy strand that includes the gene fusion sequence.

### DETAILED DESCRIPTION OF THE DISCLOSURE

Overview

The present disclosure is directed to compositions and kits which facilitate the detection of structural genomic rearrangements in samples including one or more target nucleic acids. The present disclosure is also directed to methods of detecting structural genomic rearrangements, and more specifically gene fusions, utilizing an amplicon-based approach. In some embodiments, the method described herein utilizes one or more compounds of Formula (I) for amplifying gene fusions where one fusion partner is unknown. In some embodiments, amplification with the one or more compounds of Formula (I) facilitates the detection of gene fusions with or without a sequencing step. In those embodiments where a sequencing step is utilized, such sequencing requires minimal sequencing depth.

Definitions

Unless defined otherwise, technical and scientific terms used herein have the same meaning as commonly understood by a person of ordinary skill in the art. See, Sambrook et al., Molecular Cloning, A Laboratory Manual, 4th Ed. Cold Spring Harbor Lab Press (2012).

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of” “Consisting essentially of,” when used in the claims, shall have its ordinary meaning as used in the field of patent law.

As used herein, the terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as anon-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “adaptor” refers to a nucleotide sequence that may be added to another sequence in order to import additional elements and properties to that sequence. The additional elements include without limitation: barcodes, primer binding sites, capture moieties, labels, secondary structures.

As used herein, the term “aliphatic” means a straight or branched hydrocarbon chain, which may be saturated or mono- or polyunsaturated. An unsaturated, aliphatic group contains one or more double and/or triple bonds. The branches of the hydrocarbon chain may include linear chains as well as non-aromatic cyclic elements. The hydrocarbon chain may, unless otherwise stated, be of any length, and contain any number of branches. Both the main chain as well as the branches may furthermore contain heteroatoms' as for instance B, N, O, P, S, Se or Si.

As used herein, the term “barcode” refers to a nucleic acid sequence that can be detected and identified. Barcodes can generally be 2 or more and up to about 50 nucleotides long. Barcodes are designed to have at least a minimum number of differences from other barcodes in a population. Barcodes can be unique to each molecule in a sample or unique to the sample and be shared by multiple molecules in the sample. The term “multiplex identifier,” “MID” or “sample barcode” refer to a barcode that identifies a sample or a source of the sample. As such, all or substantially all, MID barcoded polynucleotides from a single source or sample will share a MID of the same sequence; while all, or substantially all (e.g., at least 90% or 99%), MID barcoded polynucleotides from different sources or samples will have a different MID barcode sequence. Polynucleotides from different sources having different MIDs can be mixed and sequenced in parallel while maintaining the sample information encoded in the MID barcode. The term “unique molecular identifier” or “UID,” refer to a barcode that identifies a polynucleotide to which it is attached. Typically, all, or substantially all (e.g., at least 90% or 99%), UID barcodes in a mixture of UID barcoded polynucleotides are unique. Barcodes can also be used as “identifying tags” for parts of the workflow. For example, a DNA molecule derived from RNA (e.g., cDNA) may be distinguished from a DNA molecule of identical sequence derived from genomic DNA by virtue of a tag attached only to cDNA during cDNA synthesis. Such barcode may be referred to as “RNA identifying tag” or simply “identifying tag.”

As used herein, the term “ctDNA” refers to free DNA released from primary tumor cells, circulating tumor cells in the blood circulation system and necrotic or apoptotic tumor cells to the peripheral blood, or any combination thereof.

As used herein, the term “DNA polymerase” refers to an enzyme that performs template-directed synthesis of polynucleotides from deoxyribonucleotides. DNA polymerases include prokaryotic Pol I, Pol II, Pol III, Pol IV and Pol V, eukaryotic DNA polymerase, archaeal DNA polymerase, telomerase and reverse transcriptase. The term “thermostable polymerase,” refers to an enzyme that is useful in exponential amplification of nucleic acids by polymerase chain reaction (PCR) by virtue of the enzyme being heat resistant. A thermostable enzyme retains sufficient activity to effect subsequent polynucleotide extension reactions and does not become irreversibly denatured (inactivated) when subjected to the elevated temperatures for the time necessary to effect denaturation of double-stranded nucleic acids.

In some embodiments, the thermostable polymerases from species Thermococcus, Pyrococcus, Sulfolobus Methanococcus and other archaeal B polymerases. In some cases, the nucleic acid (e.g., DNA or RNA) polymerase may be a modified naturally occurring Type A polymerase. A further embodiment of the present disclosure generally relates to a method wherein a modified Type A polymerase, e.g., in a primer extension, end-modification (e.g., terminal transferase, degradation, or polishing), or amplification reaction, may be selected from any species of the genus Meiothermus, Thermotoga, or Thermomicrobium. Another embodiment of the present disclosure generally pertains to a method wherein the polymerase, e.g., in a primer extension, end-modification (e.g., terminal transferase, degradation or polishing), or amplification reaction, may be isolated from any of Thermus aquaticus (Taq), Thermus thermophilus, Thermus caldophilus, or Thermus filiformis. A further embodiment of the present disclosure generally encompasses a method wherein the modified Type A polymerase, e.g., in a primer extension, end-modification (e.g., terminal transferase, degradation, or polishing), or amplification reaction, may be isolated from Bacillus stearothermophilus, Sphaerobacter thermophilus, Dictoglomus thermophilum, or Escherichia coli. In another embodiment, the present disclosure generally relates to a method wherein the modified Type A polymerase, e.g., in a primer extension, end-modification (e.g., terminal transferase, degradation, or polishing), or amplification reaction, may be a mutant Taq-E507K polymerase. Another embodiment of the present disclosure generally pertains to a method wherein a thermostable polymerase may be used to effect amplification of the target nucleic acid.

As used herein, the term “enrichment” refers to increasing the relative amount of target molecules in the plurality of molecules. Enrichment may increase the relative amount of target molecules up to total or near total exclusion of non-target molecules. Examples of enrichment of target nucleic acids include linear hybridization capture, amplification, exponential amplification (PCR) and Primer Extension Target Enrichment (PETE), see e.g., U.S. application Ser. Nos. 14/910,237, 15/228,806, 15/648,146 and International Application Ser. No. PCT/EP2018/085727.

As used herein, the term “gene fusion” refers to a change in the genome sequence as compared to the reference genome comprising a translocation wherein a portion of one gene is fused with another sequence. Some gene fusions result in a functional fusion mRNA. A subset of those gene fusions further result in a functional fusion protein. A gene fusion has a 5′-partner and a 3′-partner designated in reference to mRNA coding for the fusion protein. The 5′-fusion partner codes for the N-terminal portion of the protein while the 3′-fusion partner codes for the C-terminal portion of the protein.

As used herein, the term “heteroatom” is meant to include boron (B), oxygen (O), nitrogen (N), sulfur (S), phosphorus (P), and silicon (Si). In some embodiments, a “heterocyclic ring” may comprise one or more heteroatoms. In other embodiments, an aliphatic group may comprise or be substituted by one or more heteroatoms.

As used herein, the terms “nucleic acid” or “polynucleotide” refer to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologues, SNPs, and complementary sequences as well as the sequence explicitly indicated.

As used herein, the term “oligonucleotide,” refers to an oligomer of nucleotide or nucleoside monomer units wherein the oligomer optionally includes non-nucleotide monomer units, and/or other chemical groups attached at internal and/or external positions of the oligomer. The oligomer can be natural or synthetic and can include naturally-occurring oligonucleotides, or oligomers that include nucleosides with non-naturally-occurring (or modified) bases, sugar moieties, phosphodiester-analog linkages, and/or alternative monomer unit chiralities and isomeric structures (e.g., 5′- to 2′-linkage, L-nucleosides, α-anomer nucleosides, β-anomer nucleosides, locked nucleic acids (LNA), peptide nucleic acids (PNA)).

As used herein, the term “primer” refers to an oligonucleotide, which binds to a specific region of a single-stranded template nucleic acid molecule and initiates nucleic acid synthesis via a polymerase-mediated enzymatic reaction. Typically, a primer comprises fewer than about 100 nucleotides and preferably comprises fewer than about 30 nucleotides. A target-specific primer specifically hybridizes to a target polynucleotide under hybridization conditions. Such hybridization conditions can include, but are not limited to, hybridization in isothermal amplification buffer (20 mM Tris-HCl, 10 mM (NH₄)₂SO₄), 50 mM KCl, 2 mM MgSO₄, 0.1% TWEEN® 20, pH 8.8 at 25° C.) at a temperature of about 40 ° C. to about 70 ° C. In addition to the target-binding region, a primer may have additional regions, typically at the 5′-poriton. The additional region may include universal primer binding site or a barcode. For exponential amplification to take place, the primers must be inward-facing, i.e., hybridizing to opposite strands of the target nucleic acid with 3′-ends facing towards each other. This orientation of amplification primers is sometimes referred to as “correct orientation.” Further, for exponential amplification to take place, the primers hybridize to the target nucleic acid within a suitable distance from each other. Under standard PCR conditions, primers hybridizing to opposite strands farther than 2000 base pairs apart would not yield a sufficient amount of product. In the case of a cfDNA sample, the typical fragment size 175 base pairs apart, therefore primers hybridizing to opposite strands farther than 175 base pairs apart would typically not yield amplified product.

As used herein, the term “reference genome” and “reference genome sequence” refer to entire human genome sequence (“genome build”) released to the public and periodically updated by the National Center for Biotechnology Information (NCBI), currently build GRCh38. The reference genome is searchable by chromosome location and sequence to enable comparing a sequence from an individual sample and identifying any sequence changes in the sample.

As used herein, the term “rearranged genome” refers to a genome comprising one or more rearrangements when compared to a reference genome. It is understood that a rearranged genome also contains non-rearranged sequences at other loci not involved in rearrangements. Such loci in the rearranged genome have the same sequence as the corresponding reference genome loci. The term “rearranged genome sequence” refers to the rearranged sequence in the rearranged genome.

As used herein, the terms “read depth” or “sequencing depth” refer to the number of times a sequence has been sequenced (the depth of sequencing). As an example, read depth can be determined by aligning multiple sequencing run results and counting the start position of reads in non-overlapping windows of a certain size (for example, 100 bp). Copy number variation can be determined based on read depth using methods known in the art. For example, using a method described in Yoon et al., Genome Research 2009 September; 19(9): 1586-1592; Xie et al., BMC Bioinformatics 2009 Mar. 6; 10:80; or Medvedev et al., Nature Methods 2009 November; 6 (11 Suppl): S13-20.

As used herein, the term “sample” refers to any biological sample that comprises nucleic acid molecules, typically comprising DNA or RNA. Samples may be tissues, cells or extracts thereof, or may be purified samples of nucleic acid molecules. The term “sample” refers to any composition containing or presumed to contain target nucleic acid. Use of the term “sample” does not necessarily imply the presence of target sequence among nucleic acid molecules present in the sample. The sample can be a specimen of tissue or fluid isolated from an individual for example, skin, plasma, serum, spinal fluid, lymph fluid, synovial fluid, urine, tears, blood cells, organs and tumors, and also to samples of in vitro cultures established from cells taken from an individual, including the formalin-fixed paraffin embedded tissues (FFPET) and nucleic acids isolated therefrom. A sample may also include cell-free material, such as cell-free blood fraction that contains cell-free DNA (cfDNA) or circulating tumor DNA (ctDNA). The sample can be collected from a non-human subject or from the environment.

In some embodiments, the “sample” is a “representative sample.” In some embodiments, a representative sample a sample (or a subset of a sample) that accurately reflects the components of the entirety and, thus, the sample is an unbiased indication of the entire population. In general, this means that the different types of cells and their relative proportion or percentages within the representative sample or a portion thereof essentially accurately reflects or mimics the relative proportion or percentages of these cell types within the entire tissue specimen, generally a solid tumor or portion thereof. Sampling is the operation of securing portions of an object for subsequent analysis. Representative samples are generated in a way that a reasonably close knowledge of the object being studied can be obtained. By contrast, conventional random sampling methods, generally does not give rise to a “representative sample.” While the selection of smaller individual sub-samples from a larger sample can be biased based on the regions selected, homogenizing a large sample, e.g., an entire tumor or lymph node, results in spatially segregated elements being homogenously dispersed throughout the sample.

As used herein, the terms “sequencing” or “DNA sequencing” refer to biochemical methods for determining the order of the nucleotide bases, adenine, guanine, cytosine, and thymine, in a DNA oligonucleotide. Sequencing, as the term is used herein, can include without limitation parallel sequencing or any other sequencing method known of those skilled in the art, for example, chain-termination methods, rapid DNA sequencing methods, wandering-spot analysis, Maxam-Gilbert sequencing, dye-terminator sequencing, or using any other modern automated DNA sequencing instruments.

As used herein, the terms “target” or “target nucleic acid” refer to the nucleic acid of interest in the sample. The sample may contain multiple targets as well as multiple copies of each target.

As used herein, the term “universal primer” refers to a primer that can hybridize to a universal primer binding site. Universal primer binding sites can be natural or artificial sequences typically added to a target sequence in a non-target-specific manner.

Compositions of Matter

In one aspect of the present disclosure is a compound of Formula (I) (also referred to herein as a “linked primer”):

[Olig1]—([R¹]ₒ—[R²]ₚ)_{q}—[L¹]ₜ—[Z]—[L²]ᵤ—[W]ᵥ—[Olig2] (I),

wherein
o is 0 or 1;
p is 0 or 1;
q is 0 or 1;
t is 0, 1 or 2;
u is 0, 1 or 2;
v is 0 or 1;
R¹ is an oligonucleotide having between about 1 and about 24 nucleotides;
R² is a substituted or unsubstituted, saturated or unsaturated, linear or cyclic aliphatic group having between 2 and about 48 carbon atoms, optionally substituted with one or more heteroatoms selected from O, N, or S;
L¹ and L² are independently a substituted or unsubstituted, saturated or unsaturated, linear or cyclic aliphatic group having between 1 and about 16 carbon atoms, optionally including with one or more heteroatoms selected from O, N, or S, and optionally including one or more carbonyl groups;
Z is a moiety selected from a triazole, a dihydropyridazine, a phosphate linkage, an amide linkage, a thioether linkage, an isooxazoline, a hydrozone, an oxime ether, and a chloro-s-triazine linkage;
W is a substituted or unsubstituted, saturated or unsaturated, aliphatic or aromatic group having between 1 and about 12 carbon atoms, optionally substituted with one or more heteroatoms selected from O, N, S, provided that W includes at least one photocleavable, enzymatically cleavable, chemically cleavable, or pH sensitive group;
Olig1 is an oligonucleotide comprising between about 1 and about 30 nucleotides; and
Olig2 is an oligonucleotide comprising between about 1 and about 30 nucleotides.

When a group is described as being “substituted or unsubstituted,” if substituted, the substituent(s) may be selected from one or more of the indicated substituents. If no substituents are indicated, it is meant that the indicated “substituted” group may be substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, cyanate, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, an ether, amino (e.g. a mono-substituted amino group or a di-substituted amino group), and protected derivatives thereof Any of the above groups may include one or more heteroatoms, including O, N, or S. For example, where a moiety is substituted with an alkyl group, that alkyl group may comprise a heteroatom selected from O, N, or S (e.g. —(CH₂—CH₂—O—CH2—CH₃)).

In some embodiments, Olig1 comprises between about 1 and about 24 nucleotides. In other embodiments, Olig1 comprises between about 1 and about 20 nucleotides. In other embodiments, Olig1 comprises between about 1 and about 16 nucleotides. In yet other embodiments, Olig1 comprises between about 1 and about 12 nucleotides. In yet other embodiments, Olig1 comprises between about 2 and about 16 nucleotides. In yet other embodiments, Olig1 comprises between about 2 and about 12 nucleotides. In yet other embodiments, Olig1 comprises between about 3 and about 12 nucleotides. In yet other embodiments, Olig1 comprises between about 4 and about 12 nucleotides. In yet other embodiments, Olig1 comprises between about 3 and about 8 nucleotides. In yet other embodiments, Olig1 comprises between about 4 and about 8 nucleotides.

In some embodiments, Olig1 has a non-extendable 3′ end. In some embodiments, the 3′-end is non-extendable due to the presence of a terminator chemical structure including e.g., a dideoxynucleotide, a 2′-phosphate nucleotide as described in U.S. Pat. No. 8,163,487 or any other 3′-O-blocked reversible terminators, and 3′unblocked reversible terminator as described e.g., in U.S. Pat. App. Pub. No. 2014/0242579 or in Guo, J., et al., Four-color DNA sequencing with 3′-O-modified nucleotide reversible terminators and chemically cleavable fluorescent dideoxynucleotides, P.N.A.S. 2008 105 (27) 9145-9150.

In some embodiments, Olig1 comprises an anchor sequence capable of hybridizing to a target sequence. Said another way, at least a portion of Olig1 is capable of hybridizing to a target nucleic acid sequence. In some embodiments, the target nucleic acid sequence is a known fusion partner. Non-limiting examples of fusion partners include ALK, PPARG, BRAF, EGFR, FGFR^{1,} FGFR^{2,} FGFR^{3,} MET, NRG1, NTRK1, NTRK2, NTRK3, RET, ROS1, AXL, PDGFRA, PDGFB , ABL1, ABL2, AKT1, AKT2, AKT3, ARHGAP26, BRD3, BRD4, CRLF2, CSF1R, EPOR, ERBB2, ERBB4, ERG, ESR1, ESRRA, ETV1, ETV4, ETVS, ETV6, EWSR1, FGR, IL2RB, INSR, JAK1, JAK2, JAK3, KIT, MAML2, MAST1, MAST2, MSMB, MUSK, MYB, MYC, NOTCH1, NOTCH2, NUMBL, NUT, PDGFRB, PIK3CA, PKN1, PRKCA, PRKCB, PTK2B, RAF1, RARA, RELA, RSPO2, RSPO3, SYK, TERT, TFE3, TFEB, THADA, TMPRSS2, TSLP, TY, BCL2, BCL6, BCR, CAMTA1, CBFB, CCNB3, CCND1, CIC, CRFL2, DUSP22, EPC1, FOXO1, FUS, GLI1, GLIS2, HMGA2, JAZF1, KMT2A, MALT1, MEAF6, MECOM, MKL1, MKL2, MTB, NCOA2, NUP214, NUP98, PAXS, PDGFB, PICALM, PLAG1, RBM15, RUNX1, RUNX1T1, SS18, STAT6, TAF15, TAL1, TCF12, TCF3, TFG, TYK2, USP6, YWHAE, AR, BRCA1, BRCA2, CDKN2A, ERB84, FLT3, KRAS, MDM4, MYBL1, NF1, NOTCH4, NUTM1, PRKACA, PRKACB, PTEN, RAD51B, and RB1.

In some embodiments, at least a portion of Olig1 is perfectly complementary to the target sequence. In other embodiments, Olig1 is only partially complementary to the target sequence. In either case, Olig1 forms a stable hybrid with the known fusion partner sequence under suitable reaction conditions for primer annealing, e.g., in a buffer containing 20 mM Tris-HCl, 10 mM (NH₄)₂SO₄, 50 mM KCl, 2 mM MgSO₄, 0.1% TWEEN® 20, pH 8.8 at 25° C., or 10 mM Tris-HCl, 50 mM KCl. 1.5 mM MgCl₂, pH 8.3 at 25° C.

In some embodiments, Olig2 comprises between about 1 and about 24 nucleotides. In other embodiments, Olig2 comprises between about 1 and about 16 nucleotides. In yet other embodiments, Olig1 comprises between about 1 and about 12 nucleotides. In yet other embodiments, Olig1 comprises between about 2 and about 9 nucleotides. In some embodiments Olig2 comprises an extendable 3′ end.

In some embodiments, Olig2 comprises a random sequence (“(N)n”). In some embodiments, the length of the random sequence can be 3, 4, 5, 6, 7, 8 or 10 or more nucleotides. To select the appropriate length of the random sequence, one of skill in the art would seek a sequence with a melting temperature (Tm) enabling a stable hybrid to form under the conditions used for hybridization of the anchor sequence. In other embodiments, Olig2 comprises a single repeating nucleotide e.g. a polyT oligonucleotide. In some embodiments, Olig2 is extended through the fusion breakpoint to form a copy strand comprising a portion of the upstream fusion partner, the fusion breakpoint and a portion of the downstream fusion partner. In some embodiments, the copy strand is used for further analysis e.g., by amplification and (or) sequencing.

In some embodiments, a portion of Olig2 is not capable of hybridizing to a target sequence. In some embodiments, the 5′-portion of Olig2 may include elements such as universal primer binding sites, platform-specific sequencing primer binding sites, barcodes (sample barcodes or molecular barcodes), or other tag sequences designed by the user. In some embodiments, the tag distinguishing RNA starting material from DNA starting material as further explained herein.

As noted above, in some embodiments, R¹ may be an oligonucleotide having between about 1 and about 16 nucleotides. In other embodiments, R¹ includes an oligonucleotide having between about 1 and about 12 nucleotides. In yet other embodiments, R¹ includes an oligonucleotide having between about 1 and about 8 nucleotides. In other embodiments, R¹ has a molecular weight ranging from between about 350 g/mol to about 5200 g/mol. In other embodiments, R¹ has a molecular weight ranging from between about 650 g/mol to about 300 g/mol.

As noted above, in some embodiments R² may be a substituted or unsubstituted, saturated or unsaturated, linear or cyclic aliphatic group having between 2 and about 48 carbon atoms, optionally substituted with one or more heteroatoms selected from O, N, or S, and optionally including one or more carbonyl groups. In other embodiments, R² includes a substituted or unsubstituted, saturated or unsaturated, linear or cyclic aliphatic group having between 2 and about 32 carbon atoms, optionally substituted with one or more heteroatoms selected from O, N, or S, and optionally including one or more carbonyl groups. In yet other embodiments, R² includes a substituted or unsubstituted, saturated or unsaturated, linear or cyclic aliphatic group having between 2 and about 28 carbon atoms, optionally substituted with one or more heteroatoms selected from O, N, or S, and optionally including one or more carbonyl groups.

In further embodiments, R² may be a substituted or unsubstituted, saturated or unsaturated, linear or cyclic aliphatic group having between 2 and about 24 carbon atoms, optionally substituted with one or more heteroatoms selected from O, N, or S, and optionally including one or more carbonyl groups. In yet further embodiments, R² includes a substituted or unsubstituted, saturated or unsaturated, linear or cyclic aliphatic group having between 2 and about 20 carbon atoms, optionally substituted with one or more heteroatoms selected from O, N, or S, and optionally including one or more carbonyl groups. In even further embodiments, R² includes a substituted or unsubstituted, saturated or unsaturated, linear or cyclic aliphatic group having between 2 and 1 about 6 carbon atoms, optionally substituted with one or more heteroatoms selected from O, N, or S, and optionally including one or more carbonyl groups. In yet even further embodiments, R² includes a substituted or unsubstituted, saturated or unsaturated, linear or cyclic aliphatic group having between 2 and about 12 carbon atoms, optionally substituted with one or more heteroatoms selected from O, N, or S. In some embodiments, the one or more carbonyl groups may be a ketone, an amide, or a carboxyl. In other embodiments, R² includes no carbonyl groups.

In some embodiments, R² includes a moiety having Formula (IVA):

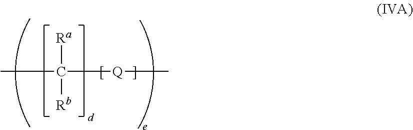

wherein d and e are integers each independently ranging from 1 to 32; Q is a bond, O, S, N(R^{c})(R^{d}) or a quaternary amine (N⁺H(R^{c})(R^{d})); R^{a} and R^{b} are independently H, a C₁-C₄ alkyl group, F, Cl, or N(^{Rc})(R^{d}); and R^{c} and R^{d} are independently CH₃ or H.

In some embodiments, d and e are integers each independently ranging from 2 to 18. In some embodiments, e ranges from 1 to 10. In other embodiments, e ranges from 1 to 8. In yet other embodiments, e ranges from 2 to 6. In yet other embodiments, e ranges from 2 to 4. In some embodiments, d is an integer ranging from 1 to 8, and e is an integer ranging from 2 to 16. In other embodiments, d is an integer ranging from 2 to 8, and e is an integer ranging from 2 to 12. In other embodiments, d is 2 or 3, and e is an integer ranging from 2 to 12. In other embodiments, d is 2 or 3, and e is an integer ranging from 2 to 8. In other embodiments, d is 2 or 3, and e is an integer ranging from 2 to 6. In other embodiments, d is 2 or 3, and e is an integer ranging from 2 to 4. In some embodiments, at least one of R^{a} or R^{b} is —CH₃.

In other embodiments, R² includes a moiety having Formula (IVB):

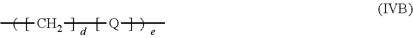

wherein d and e are integers each independently ranging from 1 to 32; Q is a bond, O, S, or N(R^{c})(R^{d}); and R^{c} and R^{d} are independently CH₃ or H.

In some embodiments, e ranges from 1 to 10. In other embodiments, e ranges from 1 to 8. In yet other embodiments, e ranges from 2 to 6. In yet other embodiments, e ranges from 2 to 4. In other embodiments, Q is 0. In some embodiments, d is an integer ranging from 1 to 8, and e is an integer ranging from 2 to 16. In other embodiments, d is an integer ranging from 2 to 8, and e is an integer ranging from 2 to 12. In other embodiments, d is 2 or 3, and e is an integer ranging from 2 to 12. In other embodiments, d is 2 or 3, and e is an integer ranging from 2 to 8. In other embodiments, d is 2 or 3, and e is an integer ranging from 2 to 6. In other embodiments, d is 2 or 3, and e is an integer ranging from 2 to 4.

In yet other embodiments, R² includes a moiety having Formula (IVC):

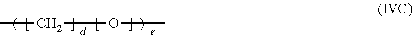

wherein d and e are integers each independently ranging from 1 to 32. In some embodiments, e ranges from 1 to 10. In other embodiments, e ranges from 1 to 8. In yet other embodiments, e ranges from 2 to 6. In yet other embodiments, e ranges from 2 to 4. In some embodiments, d ranges from 1 to 4, and e ranges from 1 to about 8. In some embodiments, d is an integer ranging from 1 to 8, and e is an integer ranging from 2 to about 16. In other embodiments, d is an integer ranging from 2 to 8, and e is an integer ranging from 2 to about 12. In other embodiments, d is 2 or 3, and e is an integer ranging from 2 to 12. In other embodiments, d is 2 or 3, and e is an integer ranging from 2 to 8. In other embodiments, d is 2 or 3, and e is an integer ranging from 2 to 6. In other embodiments, d is 2 or 3, and e is an integer ranging from 2 to 4.

In some embodiments, the R² includes a solubilizing group. In some embodiments, the solubilizing group is as polyethylene glycol (PEG) group or a polypropylene glycol group. In yet other embodiments, the Linkers comprises between about 2 and about 8 PEG groups or polypropylene glycol groups. In yet other embodiments, the Linkers comprise about 6 PEG groups or polypropylene glycol groups. In yet other embodiments, the Linkers comprise about 4 PEG groups polypropylene glycol groups. In yet other embodiments, the Linkers comprise 2 PEG groups or polypropylene glycol groups.

As noted above, in some embodiments L¹ may be a substituted or unsubstituted, saturated or unsaturated, linear or cyclic aliphatic group having between 1 and about 16 carbon atoms, optionally substituted with one or more heteroatoms selected from O, N, or S, and optionally including one or more carbonyl groups. In other embodiments, L¹ includes a substituted or unsubstituted, saturated or unsaturated, linear or cyclic aliphatic group having between 1 and about 12 carbon atoms, optionally substituted with one or more heteroatoms selected from O, N, or S, and optionally including one or more carbonyl groups. In yet other embodiments, L¹ includes a substituted or unsubstituted, saturated or unsaturated, linear or cyclic aliphatic group having between 1 and about 8 carbon atoms, optionally substituted with one or more heteroatoms selected from O, N, or S, and optionally including one or more carbonyl groups.

In further embodiments, L¹ includes a substituted or unsubstituted, saturated or unsaturated, linear or cyclic aliphatic group having between 1 and about 6 carbon atoms, optionally substituted with one or more heteroatoms selected from O, N, or S, and optionally including one or more carbonyl groups. In even further embodiments, L¹ includes a substituted or unsubstituted, saturated or unsaturated, linear or cyclic aliphatic group having between 1 and about 4 carbon atoms, optionally substituted with one or more heteroatoms selected from O, N, or S, and optionally including one or more carbonyl groups. In some embodiments, the group L¹ nay include one or more solubilizing groups, e.g. PEG groups. In some embodiments, the carbonyl group is selected from a ketone, an amide, and a carboxyl. In some embodiments, the group L¹ includes a ketone. In some embodiments, the group L¹ includes an amide.

In some embodiments, the group —([R¹]ₒ—[R²]ₚ)_{q}— has a length ranging from between about 15 Angstroms to about 1000 Angstroms. In other embodiments, the group —([R¹]ₒ—[R²]ₚ)_{q}—has a length ranging from between about 15 Angstroms to about 500 Angstroms. In yet other embodiments, the group —([R¹]ₒ—[R²]ₚ)_{q}— has a length ranging from between about 15 Angstroms to about 400 Angstroms. In yet other embodiments, —([R¹]ₒ—[R²]ₚ)_{q}— has a length ranging from between about 15 Angstroms to about 300 Angstroms. In yet other embodiments, the group —([R¹]ₒ—[R²]ₚ)_{q}— has a length ranging from between about 15 Angstroms to about 250 Angstroms. In yet other embodiments, the group —([R¹]ₒ—[R²]ₚ)_{q}— has a length ranging from between about 15 Angstroms to about 200 Angstroms. In yet other embodiments, the group —([R¹]ₒ—[R²]ₚ)_{q}— has a length ranging from between about 15 Angstroms to about 150 Angstroms. In yet other embodiments, the group —([R¹]ₒ—[R²]ₚ)_{q}— has a length ranging from between about 15 Angstroms to about 100 Angstroms. In yet other embodiments, —([R¹]ₒ—[R²]ₚ)_{q}— has a length ranging from between about 15 Angstroms to about 50 Angstroms. In yet other embodiments, —([R¹]ₒ—[R²]ₚ)_{q}— has a length ranging from between about 20 Angstroms to about 40 Angstroms.

In some embodiments, o+p=1, and q is 1. In other embodiments, o is 1, p is 0, and q is 1. In yet other embodiments, o is 0, p is 1, and q is 1. In yet other embodiments, o is 0, p is 1, and q is 2.

In some embodiments, o is 1, p is 0, and q is 1, and R¹ comprises between about 1 and about 12 nucleotides. In some embodiments, o is 1, p is 0, and q is 1, and R¹ comprises between about 1 and about 8 nucleotides. In some embodiments, o is 1, p is 0, and q is 1, and R¹ comprises between about 1 and about 6 nucleotides.

In some embodiments, o is 0, p and q are both 1, and R² includes a solubilizing group. In some embodiments, o is 0 and p and q are both 1, and R² includes at least one PEG group. In some embodiments, o is 0 and p and q are both 1, and R² includes at least 4 PEG groups. In some embodiments, o is 0 and p and q are both 1, and R² includes at least 6 PEG groups. In some embodiments, o is 0 and p and q are both 1, and R² includes at least 8 PEG groups. In some embodiments, o is 0 and p and q are both 1, and R² includes at least 10 PEG groups. In some embodiments, o is 0 and p and q are both 1, and R² includes at least 12 PEG groups. In some embodiments, o is 0 and p and q are both 1, and R² includes at least 16 PEG groups.

In some embodiments, o is 0 and p and q are both 1, and R² includes a group having Formula (IVB). In some embodiments, o is 0 and p and q are both 1, and R² includes a group having Formula (IVB), and where e ranges from 1 to 16. In some embodiments, o is 0 and p and q are both 1, and R² includes a group having Formula (IVB), and where e ranges from 1 to 12. In some embodiments, o is 0 and p and q are both 1, and R² includes a group having Formula (IVB), and where e ranges from 1 to 8.

In some embodiments, o is 0 and p and q are both 1, and R² includes a group having Formula (IVB), d is 2 or 3, and where e ranges from 1 to 16. In some embodiments, o is 0 and p and q are both 1, and R² includes a group having Formula (IVB), d is 2 or 3, and where e ranges from 1 to 12. In some embodiments, o is 0 and p and q are both 1, and R² includes a group having Formula (IVB), d is 2 or 3, and where e ranges from 1 to 10. In some embodiments, o is 0 and p and q are both 1, and R² includes a group having Formula (IVB), d is 2 or 3, and where e ranges from 1 to 8. In some embodiments, o is 0 and p and q are both 1, and R² includes a group having Formula (IVB), d is 2 or 3, and where e ranges from 1 to 6. In some embodiments, o is 0 and p and q are both 1, and R² includes a group having Formula (IVB), d is 2 or 3, and where e ranges from 1 to 4.

In some embodiments, o is 0 and p and q are both 1, and R² includes a group having Formula (IVB), d is 2, and where e ranges from 1 to 12. In some embodiments, o is 0 and p and q are both 1, and R² includes a group having Formula (IVB), d is 2, and where e ranges from 1 to 10. In some embodiments, o is 0 and p and q are both 1, and R² includes a group having Formula (IVB), d is 2, and where e ranges from 1 to 8. In some embodiments, o is 0 and p and q are both 1, and R² includes a group having Formula (IVB), d is 2, and where e ranges from 1 to 6. In some embodiments, o is 0 and p and q are both 1, and R² includes a group having Formula (IVB), d is 2, and where e ranges from 1 to 4.

In some embodiments, o is 0 and p and q are both 1, and R² includes a group having Formula (IVC). In some embodiments, o is 0 and p and q are both 1, and R² includes a group having Formula (IVC), and where e ranges from 1 to 16. In some embodiments, o is 0 and p and q are both 1, and R² includes a group having Formula (IVC), and where e ranges from 1 to 12. In some embodiments, o is 0 and p and q are both 1, and R² includes a group having Formula (IVC), and where e ranges from 1 to 8. In some embodiments, o is 0 and p and q are both 1, and R² includes a group having Formula (IVC), and where e ranges from 1 to 4. In some embodiments, o is 0 and p and q are both 1, and R² includes a group having Formula (IVC), d is 2 or 3, and where e ranges from 1 to 16. In some embodiments, o is 0 and p and q are both 1, and R² includes a group having Formula (IVC), d is 2 or 3, and where e ranges from 1 to 12. In some embodiments, o is 0 and p and q are both 1, and R² includes a group having Formula (IVC), d is 2 or 3, and where e ranges from 1 to 10. In some embodiments, o is 0 and p and q are both 1, and R² includes a group having Formula (IVC), d is 2 or 3, and where e ranges from 1 to 8. In some embodiments, o is 0 and p and q are both 1, and R² includes a group having Formula (IVC), d is 2 or 3, and where e ranges from 1 to 6. In some embodiments, o is 0 and p and q are both 1, and R² includes a group having Formula (IVC), d is 2 or 3, and where e ranges from 1 to 4.

As noted above, in some embodiments L² may be a substituted or unsubstituted, saturated or unsaturated, linear or cyclic aliphatic group having between 1 and about 16 carbon atoms, optionally substituted with one or more heteroatoms selected from O, N, or S, and optionally including one or more carbonyl groups. In other embodiments, L² includes a substituted or unsubstituted, saturated or unsaturated, linear or cyclic aliphatic group having between 1 and about 12 carbon atoms, optionally substituted with one or more heteroatoms selected from O, N, or S, and optionally including one or more carbonyl groups. In yet other embodiments, L² includes a substituted or unsubstituted, saturated or unsaturated, linear or cyclic aliphatic group having between 1 and about 8 carbon atoms, optionally substituted with one or more heteroatoms selected from O, N, or S, and optionally including one or more carbonyl groups.

In further embodiments, L² includes a substituted or unsubstituted, saturated or unsaturated, linear or cyclic aliphatic group having between 1 and about 6 carbon atoms, optionally substituted with one or more heteroatoms selected from O, N, or S, and optionally including one or more carbonyl groups. In even further embodiments, L² includes a substituted or unsubstituted, saturated or unsaturated, linear or cyclic aliphatic group having between 1 and about 4 carbon atoms, optionally substituted with one or more heteroatoms selected from O, N, or S, and optionally including one or more carbonyl groups. In some embodiments, the group L² nay include one or more solubilizing groups, e.g. PEG groups. In some embodiments, the carbonyl group is selected from a ketone, an amide, and a carboxyl. In some embodiments, the group L² includes a ketone. In some embodiments, the group L² includes an amide.

In some embodiments, the compound of Formula (I) includes a cleavage site for cleaving the compound of Formula (I). In some embodiments, the cleavage site is located within Olig2. In these embodiments, v is 0 and no W group is present. In these embodiments, Olig2 may include, for example, at least one uracil-containing nucleotide. In some embodiments, the uracil-containing nucleotide can be cleaved by adding Uracil-N-DNA glycosylase (UNG), optionally in the presence of primary amines as described in U.S. Pat. No. 8,669,061. In some embodiments, cleavage is performed by a combination of a glycosylase and an endonuclease, e.g., by a mixture of Uracil DNA glycosylase (UDG) and the DNA glycosylase-lyase Endonuclease VIII.

In other embodiments, the cleavage site is located external to Olig2, such as in the group W. In some embodiments, and as noted above, W includes a substituted or unsubstituted, saturated or unsaturated, aliphatic or aromatic group having between 1 and about 12 carbon atoms, optionally substituted with one or more heteroatoms selected from O, N, S, provided that W includes a photocleavable, enzymatically cleavable, chemically cleavable, or pH sensitive group. In other embodiments, W includes a substituted or unsubstituted, saturated or unsaturated, aliphatic or aromatic group having between 1 and about 8 carbon atoms, optionally substituted with one or more heteroatoms selected from O, N, S, provided that W includes a photocleavable, enzymatically cleavable, chemically cleavable, or pH sensitive group.

In yet other embodiments, W includes a substituted or unsubstituted, saturated or unsaturated, aliphatic or aromatic group having between 1 and about 6 carbon atoms, optionally substituted with one or more heteroatoms selected from O, N, S, provided that W includes at least one photocleavable, enzymatically cleavable, chemically cleavable, or pH sensitive group. In further embodiments, W includes a substituted or unsubstituted, saturated or unsaturated, aliphatic or aromatic group having between 1 and about 4 carbon atoms, optionally substituted with one or more heteroatoms selected from O, N, S, provided that W includes at least one photocleavable, enzymatically cleavable, chemically cleavable, or pH sensitive group.

In some embodiments, W includes at least one photocleavable moiety. In some embodiments, the photocleavable moiety may be cleaved upon exposure to an electromagnetic radiation source having a wavelength of between about 200 nm to about 400 nm (UV) or between about 400 nm to about 800 nm (visible). Examples of suitable photocleavable moieties include, but are not limited to, arylcarbonylmethyl groups (e.g. 4-acetyl-2-nitrobenzyl, dimethylphenacyl (DMP)); 2-(alkoxymethyl)-5-methyl-α-chloroacetophenones, 2,5-dimethylbenzoyl oxiranes, benzoin groups (e.g. 3′,5′-dimethoxybenzoin (DMB)), o-nitrobenzyl groups (e.g. 1-(2-nitrophenyl)ethyl (NPE), 1-(methoxymethyl)-2-nitrobenzene, 4,5-dimethoxy-2-nitrobenzyl (DMNB), α-carboxynitrobenzyl (α-CNB)); o-nitro-2-phenethyloxycarbonyl groups (e.g. 1-(2-nitrophenyl)ethyloxycarbonyl and 2-nitro-2-phenethyl derivatives); o-nitroanilides (e.g. acylated 5-bromo-7-nitroindolines); coumarin-4-yl-methyl groups (e.g. 7-methoxycoumarin derivatives); 9-substituted xanthenes, and arylmethyl groups (e.g. o-hydroxyarylmethyl groups).

In some embodiments, the at least one photocleavable moiety may be cleaved upon exposure to an electromagnetic radiation source having a wavelength of between about 700 nm to about 1000 nm. Suitable near-infrared photocleavable groups include cyanine groups, including C4 dialkylamine-substituted heptamethine cyanines.

In some embodiments, W includes at least one chemically cleavable moiety. In some embodiments, the chemically cleavable moiety is a group which may be chemically cleaved by different chemical reactants, including reducing agents or by induced changes in pH (e.g. cleavage of the group at a pH of less than 7). Non-limiting examples of chemically cleavable moieties include disulfide-based groups; diazobenzene groups (e.g. 2-(2-alkoxy-4-hydroxy-phenylazo); benzoic acid scaffolds; ester bond-based groups; and acidic sensitive groups (e.g. a dialkoxydiphenylsilane group or acylhydrazone group). Electrophilically cleaved groups (e.g. p-alkoxybenzyl esters and p-alkoxybenzyl amides) are believed to be cleaved by protons and include cleavages sensitive to acids.

In some embodiments, W includes at least one enzymatically cleavable moiety. In some embodiments, the enzymatically cleavable moiety may be cleaved by, for example, trypsin cleavable groups and V8 protease cleavable groups. In some embodiments, the at least one enzymatically cleavable moiety may be enzymatically cleaved by one of a USER enzyme, uracil-N-glycosylase, an RNase A, a beta-glucuronidase, a beta-galactosidase, or a TEV-protease.

In another aspect of the present disclosure is a compound having Formula (II):

[Olig1]—([R¹]ₒ—[R²]ₚ)_{q}—[L¹]ₜ—[X] (II),

wherein
o is 0 or 1;
p is 0 or 1;
q is 1 or 2;
t is 0, 1 or 2;
R¹ is an oligonucleotide having between about 1 and about 24 nucleotides;
R² is a substituted or unsubstituted, saturated or unsaturated, linear or cyclic aliphatic group having between 2 and about 48 carbon atoms, optionally substituted with one or more heteroatoms selected from O, N, or S;
L¹ is a substituted or unsubstituted, saturated or unsaturated, linear or cyclic aliphatic group having between 1 and about 16 carbon atoms, optionally substituted with one or more heteroatoms selected from O, N, or S, and optionally including one or more carbonyl groups;
X is a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, an amino group, or a phosphoramidite; and
Olig1 is an oligonucleotide comprising between about 1 and about 30 nucleotides.

In some embodiments, Olig1 comprises between about 1 and about 24 nucleotides. In other embodiments, Olig1 comprises between about 1 and about 16 nucleotides. In some embodiments, Olig1 has a non-extendable 3′ end.

In some embodiments, Olig1 comprises an anchor sequence capable of hybridizing to a known fusion partner. Non-limiting examples of fusion partners include ALK, PPARG, BRAF, EGFR, FGFR^{1,} FGFR^{2,} FGFR^{3,} MET, NRG1, NTRK1, NTRK2, NTRK3, RET, ROS1, AXL, PDGFRA, PDGFB , ABL1, ABL2, AKT1, AKT2, AKT3, ARHGAP26, BRD3, BRD4, CRLF2, CSF1R, EPOR, ERBB2, ERBB4, ERG, ESR1, ESRRA, ETV1, ETV4, ETV5, ETV6, EWSR1, FGR, IL2RB, INSR, JAK1, JAK2, JAK3, KIT, MAML2, MAST1, MAST2, MSMB, MUSK, MYB, MYC, NOTCH1, NOTCH2, NUMBL, NUT, PDGFRB, PIK3CA, PKN1, PRKCA, PRKCB, PTK2B, RAF1, RARA, RELA, RSPO2, RSPO3, SYK, TERT, TFE3, TFEB, THADA, TMPRSS2, TSLP, TY, BCL2, BCL6, BCR, CAMTA1, CBFB, CCNB3, CCND1, CIC, CRFL2, DUSP22, EPC1, FOXO1, FUS, GLI1, GLIS2, HMGA2, JAZF1, KMT2A, MALT1, MEAF6, MECOM, MKL1, MKL2, MTB, NCOA2, NUP214, NUP98, PAXS, PDGFB, PICALM, PLAG1, RBM15, RUNX1, RUNX1T1, SS18, STAT6, TAF15, TAL1, TCF12, TCF3, TFG, TYK2, USP6, YWHAE, AR, BRCA1, BRCA2, CDKN2A, ERB84, FLT3, KRAS, MDM4, MYBL1, NF1, NOTCH4, NUTM1, PRKACA, PRKACB, PTEN, RAD51B, and RB1.

In another aspect of the present disclosure is a compound having Formula (II):

[Y]—[L²]ᵤ—[W]ᵥ—[Olig2] (III),

wherein
u is 0, 1 or 2;
v is 0 or 1;
Y is a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, an amino group, or a phosphoramidite;
L² is a substituted or unsubstituted, saturated or unsaturated, linear or cyclic aliphatic group having between 1 and 16 carbon atoms, optionally including with one or more heteroatoms selected from O, N, or S, and optionally including one or more carbonyl groups;
W is a substituted or unsubstituted, saturated or unsaturated, aliphatic or aromatic group having between 1 and 12 carbon atoms, optionally substituted with one or more heteroatoms selected from O, N, S, provided that W includes at least one photocleavable, enzymatically cleavable, chemically cleavable, or pH sensitive group; and
Olig2 is an oligonucleotide comprising between about 1 and about 30 nucleotides.

In some embodiments, Olig2 comprises between about 1 and about 24 nucleotides. In other embodiments, Olig2 comprises between about 1 and about 16 nucleotides. In yet other embodiments, Olig2 comprises between about 1 and about 12 nucleotides. In some embodiments Olig2 comprises an extendable 3′ end. In some embodiments, Olig2 comprises a random sequence. In other embodiments, Olig2 comprises a single repeating nucleotide e.g. a polyT oligonucleotide.

Preparation of the Compounds of Formulas (I), (II), and (III)

The skilled artisan will appreciate that the compounds of Formula (II) and Formula (III) may be reacted with each other to form a compound having Formula (I). In some embodiments, a group Z of Formula (I) is determined by the X and Y groups of Formulas (II) and (III), respectively. Table 2 sets forth the X and Y groups of Formulas (II) and (III) and the resulting group Z of a formed compound having Formula (I).

| X | Y | Z |
|---|---|---|
| Alkyne | Azide | 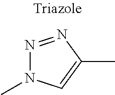 |
| Azide | Alkyne | 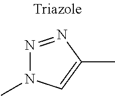 |
| diarylcyclooctyne (“DBCO”) | Azide | 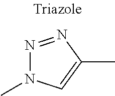 |
| Alkene | Tetrazine | Dihydropyridazine |
| Trans-cyclooctene | Tetrazine | Dihydropyridazine |
| (“TCO”) | | |
| Maleimide | Thiol | 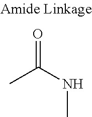 |
| DBCO | 1,3-Nitrone | Isoxazoline |
| Aldehyde or ketone | Hydrazine | Hydrazone |
| Aldehyde or ketone | Hydroxylamine | Oxime ether |
| Azide | DBCO | 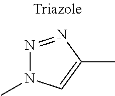 |
| Tetrazine | TCO | Dihydropyridazine |
| Thiol | Maleimide | 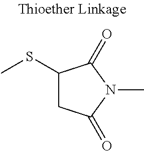 |
| 1,3-Nitrone | DBCO | Isoxazoline |
| Hydrazine | Aldehyde or ketone | Hydrazone |
| Hydroxylamine | Aldehyde or ketone | Oxime ether |
| Tetrazine | Alkene | Dihydropyridazine |
| Amino (in the | Amino (in the | Chloro-s-Triazine Linkage |
| presence of s- | presence of s- | |
| Trichlorotriazine) | Trichlorotriazine) | |

In some embodiments, the groups Olig1 and Olig2 of Formulas (II) and (III) are prepared according to methods known to those of ordinary skill in the art. In some embodiments, the groups Olig1 and Olig2 are synthesized using solid-phase synthesis techniques employing phosphoramidite chemistry, (see, e.g., Protocols for Oligonucleotides and Analogs, Agrawal, S., ed., Humana Press, Totowa, N.J., 1993, hereby incorporated by reference in its entirety). Other methods of synthesizing Olig1, Olig2, and/or the compounds of Formulas (II) and (III) are described in U.S. Pat. Nos. 5,955,591, 6,057,431, 8,889,843, and 6,124,445; and in U.S. Patent Publication Nos. 2008/0119645 and 2003/0153743, the disclosures of which are hereby incorporated by reference herein in their entireties.

In some embodiments, the first step in such a process is the attachment of a first monomer or higher order subunit containing a protected 5′-hydroxyl to a solid support, usually through a linker, using standard methods and procedures known in the art. See for example, Oligonucleotides and Analogues A Practical Approach, Ekstein, F. Ed., IRL Press, N.Y, 1991. The support-bound monomer or higher order first synthon is then treated to remove the 5′-protecting group. In some embodiments, this is accomplished by treatment with acid. In some embodiments, the solid support bound monomer is then reacted with a phosphoramidite to form a phosphite linkage. In some embodiments, the phosphite-containing compounds are oxidized to produce compounds having a desired internucleotide linkage. In some embodiments, the choice of oxidizing agent will determine whether the phosphite linkage will be oxidized to, for example, a phosphotriester, thiophosphotriester, or a dithiophosphotriester linkage.

In some embodiments, a capping step is performed either prior to or after oxidation of the phosphite triester, thiophosphite triester, or dithiophosphite triester. In some embodiments, the capping step involves attachment of a “cap” moiety to oligonucleotide chains that have not reacted in a given coupling cycle. The cap moiety, in some embodiments, is reactive with the terminal portion of oligonucleotides that did not participate in the coupling cycle but is not reactive with oligonucleotides that did participate and, moreover, is not itself reactive with the coupling reagents.

Further treatment of the oxidized oligomer with an acid removes the 5′-hydroxyl protecting group, and thus transforms the solid support bound oligomer into a further compound which may be subsequently reacted to begin the next synthetic iteration. This process is repeated until an oligomer of desired length is produced.

In some embodiments, the compounds of Formula (II) and (III) may be reacted to form a compound of Formula (I). In these embodiments, a 5′ to 5′ linkage may be formed between the compounds of Formula (II) and those of Formula (II). In some embodiments, compounds having Formula (II) are synthesized, such as using the procedures described above, in the 3′ to 5′ direction. Such a synthesis may be carried out using 3′ amidites.

The compounds of Formula (III) may also be synthesized in a similar manner but using 5′ amidites instead of 3′ amidites. Non-limiting examples of 5′ amidites are set forth below. In this manner, the compounds of Formula (III) may be synthesized in the 5′ to 3′ direction. In some embodiments, the compounds of Formulas (II) and (III) may be linked through a phosphate linkage.

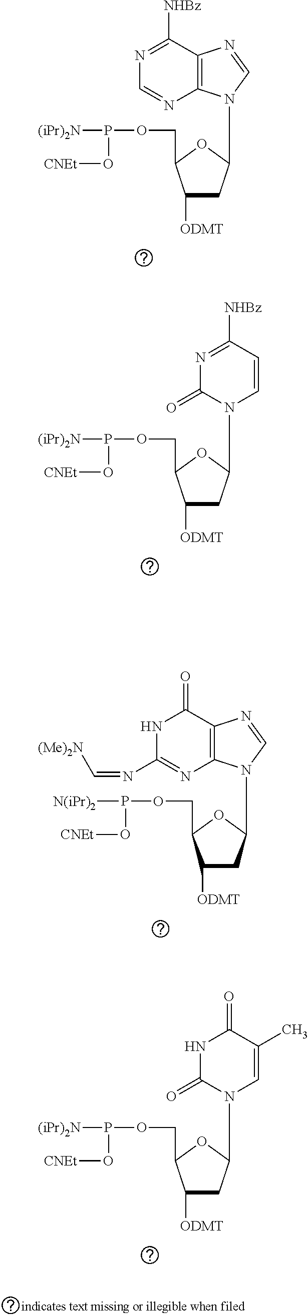

In some embodiments, the compounds of Formula (II) and Formula (III) may be reacted with each other using “click chemistry.” “Click chemistry” is a chemical philosophy, independently defined by the groups of Sharpless and Meldal, that describes chemistry tailored to generate substances quickly and reliably by joining small units together. “Click chemistry” has been applied to a collection of reliable and self-directed organic reactions (Kolb, H. C.; Finn, M. G.; Sharpless, K. B. Angew). Chem. Int. Ed. 2001, 40, 2004-2021). For example, the identification of the copper catalyzed azide-alkyne [3+2] cycloaddition as a highly reliable molecular connection in water (Rostovtsev, V. V.; et al. Angew. Chem. Int. Ed. 2002, 41, 2596-2599) has been used to augment several types of investigations of biomolecular interactions (Wang, Q.; et al. J. Am. Chem. Soc. 2003, 125, 3192-3193; Speers, A. E.; et al. J. Am. Chem. Soc. 2003, 125, 4686-4687; Link, A. J.; Tirrell, D. A. J. Am. Chem. Soc. 2003, 125, 11164-11165; Deiters, A.; et al. J. Am. Chem. Soc. 2003, 125, 11782-11783). In addition, applications to organic synthesis (Lee, L. V.; et al. J. Am. Chem. Soc. 2003, 125, 9588-9589), drug discovery (Kolb, H. C.; Sharpless, K. B. Drug Disc. Today 2003, 8, 1128-1137; Lewis, W. G.; et al. Angew. Chem. Int. Ed. 2002, 41, 1053-1057), and the functionalization of surfaces (Meng, J.-C.; et al. Angew. Chem. Int. Ed. 2004, 43, 1255-1260; Fazio, F.; et al. J. Am. Chem. Soc. 2002, 124, 14397-14402; Collman, J. P.; et al. Langmuir 2004, ASAP, in press; Lummerstorfer, T.; Hoffmann, H. J. Phys. Chem. B 2004, in press) have also appeared.

In some embodiments, precursors of the compounds of Formula (II) are first modified to introduce a first member of a pair of reactive functional groups capable of participating in a “click chemistry” reaction. Likewise, in some embodiments, precursors of the compounds of Formula (III) are modified to introduce a second member of the pair of reactive functional groups capable of participating in a “click chemistry” reaction. In some embodiments, the first and second members of the pair of reactive functional groups capable of participating in a “click chemistry” reaction are identified in Table 1. In some embodiments, the “click chemistry” reaction is catalyzed with an introduced reagent. In some embodiments, the introduced reagent is Cu⁺.

TABLE 1
First and second members of reactive functional group pairs.

| Reactive Functional Group | Reactive Functional Group |
|---|---|
| on a First Member of a | on a Second Member of a |
| Pair of Click Conjugates | Pair of Click Conjugates |
| Alkyne | Azide |
| Azide | Alkyne |
| diarylcyclooctyne | Azide |
| (“DBCO”) | |
| Alkene | Tetrazine |
| Trans-cyclooctene | Tetrazine |
| (“TCO”) | |
| Maleimide | Thiol |
| DBCO | 1,3-Nitrone |
| Aldehyde or ketone | Hydrazine |
| Aldehyde or ketone | Hydroxylamine |
| Azide | DBCO |
| Tetrazine | TCO |
| Thiol | Maleimide |
| 1,3-Nitrone | DBCO |
| Hydrazine | Aldehyde or ketone |
| Hydroxylamine | Aldehyde or ketone |
| Tetrazine | Alkene |

By way of example only, a precursor to a compound of Formula (II) may be modified to introduce a primary halogen. Subsequently, sodium azide may be introduced, which reacts with the primary halogen such that the precursor to the compound of Formula (II) is converted to the azide. In some embodiments, the precursor to the compound of Formula (II) is reacted with an amidite including the primary halogen either directly or indirectly through a linker. A non-limiting example of a suitable amidite is illustrated below:

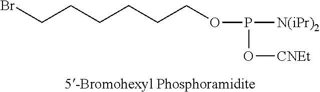

Again by way of example, a precursor to a compound of Formula (III) may be modified (such as with an amidite) to introduce a moiety which is reactive with the azide of Formula (II), such as a moiety including an alkyl group. Non-limiting examples of suitable amidites are provided below:

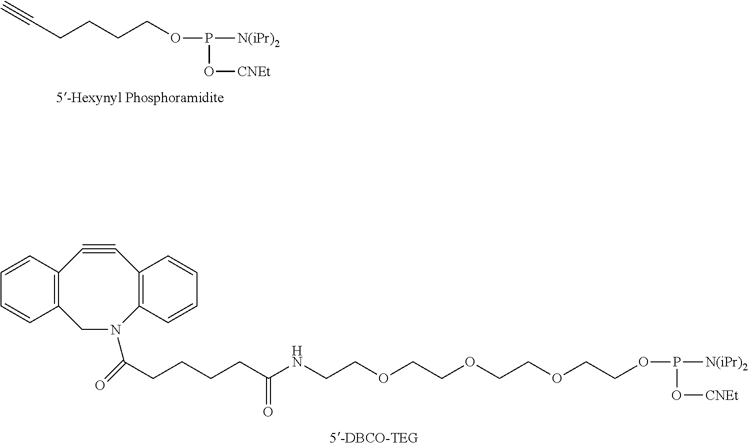

Another suitable reagent is DBCO-PEG-Phosphoramidite, such as DBCO-PEG4-Phosphoramidite:

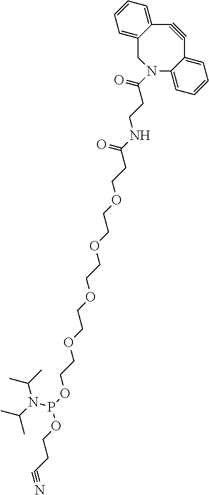 [SMILES: CC(C)N(C(C)C)P(OCCC#N)OCCOCCOCCOCCOCCC(=O)NCCC(=O)N1Cc2ccccc2C#Cc2ccccc21]

The resulting compounds of Formula (II) and (III), each bearing a member of the reactive groups capable of participating in a “click chemistry” reaction, are then allowed to react with each other to form the 5′ to 5′ linkage. In the example provided above, the azide and alkyne will reactive to form a triazole linkage.

In some embodiments, the compounds of Formulas (II) and (III) may each include reactive groups (X and Y, respectively) that facilitate the formation of an amide linkage between the compounds. To achieve this, in some embodiments precursors to the compounds of each of Formulas (II) and (III) may be reacted with a reagent which introduces the groups X and Y, respectively. In these embodiments, a precursor to a compound having Formula (II) is modified with an amino moiety at a 5′ end. For instance, an amidite may be introduced to a precursor of a compound having Formula (II), where the amidite includes a terminal amino moiety. Non-limiting examples of such amidite reagents include the following:

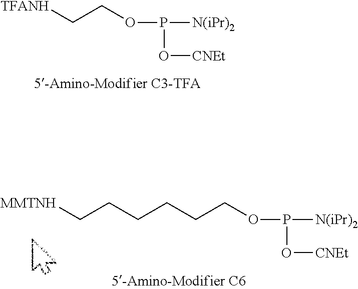

Similarly, a precursor to a compound having Formula (III) may also be modified at a 5′ end to terminate in a carboxyl group. For instance, an amidite may be introduced to a precursor of a compound having Formula (III), where the amidite includes a terminal carboxyl moiety. A non-limiting example of such an amidite reagent is:

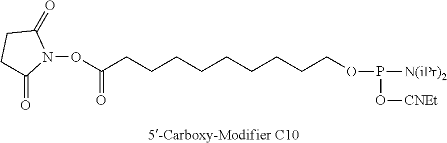

In some embodiments, the compounds of Formulas (II) and (III) may each include reactive groups (X and Y, respectively) that facilitate the formation of a thioether linkage between the compounds. To achieve this, in some embodiments precursors to the compounds of each of Formulas (II) and (III) may be reacted with a reagent which introduces the groups X and Y, respectively. In these embodiments, a precursor to a compound having Formula (II) is modified with a thiol moiety at a 5′ end. For instance, an amidite may be introduced to a precursor to a compound having Formula (II), where the amidite includes a terminal thiol moiety. Non-limiting examples of such amidite reagents include the following:

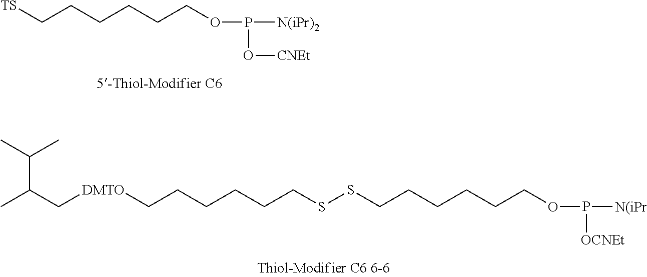

A compound having Formula (III) may also be modified at a 5′ end to terminate in a maleimide group. For instance, an amidite may be introduced to a precursor to a compound having Formula (III), where the amidite includes a terminal maleimide moiety. A non-limiting example of such an amidite reagent is:

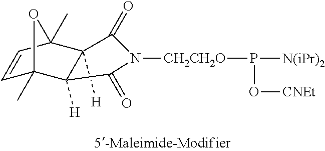

In some embodiments, the compounds of Formulas (II) and (III) may each include reactive groups (X and Y, respectively) that facilitate the formation of a triazine linkage between the compounds. To achieve this, in some embodiments precursors to the compounds of each of Formulas (II) and (III) may be reacted with a reagent which introduces the groups X and Y, respectively. In some embodiments, the triazine linkage is a chloro-s-triazine linkage. In these embodiments, a precursor to a compound having Formula (II) is modified with an amino moiety at a 5′ end. Likewise, a precursor to a compound having Formula (III) is modified with an amino moiety at a 5′ end. Non-limiting examples of suitable amidites for introducing such a 5′ amino group are set forth below:

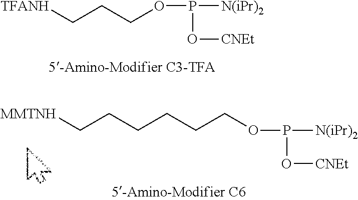

Following the modification of both the precursor to the compound of Formula (II) and the precursor to the compound of Formula (III), the formed compounds of Formula (II) and (III) are then reacted with a coupling reagent. In some embodiments, the coupling reagent is s-trichlorotriazine. This reaction is illustrated below:

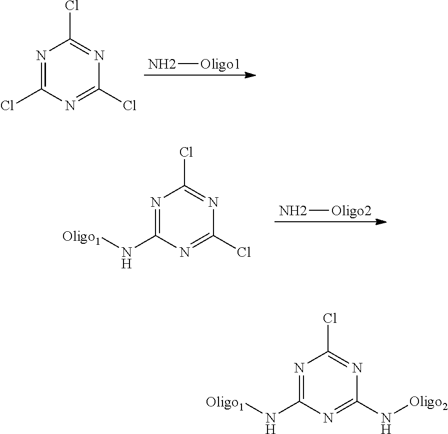

In some embodiments, any precursor of a compound of Formula (II) or (III) may be reacted to introduce a linker or spacer, such as a PEG-based linker or spacer. A non-limiting example of a suitable reagent to introduce a PEG-based linker or spacer is set forth below:

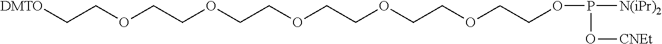

Other reagents and methods for incorporating a PEG-based linker or spacer into the precursors of the compounds of Formulas (II) and/or (III) are described in U.S. Patent Publication No. 2006/0063147, the disclosure of which is hereby incorporated by reference herein in its entirety.

In some embodiments, any precursor to a compound of Formula (II) or (III) may be reacted to introduce a linker or spacer, such as a linker or spacer including a cleavable group. Non-limiting examples of a suitable reagents are set forth below:

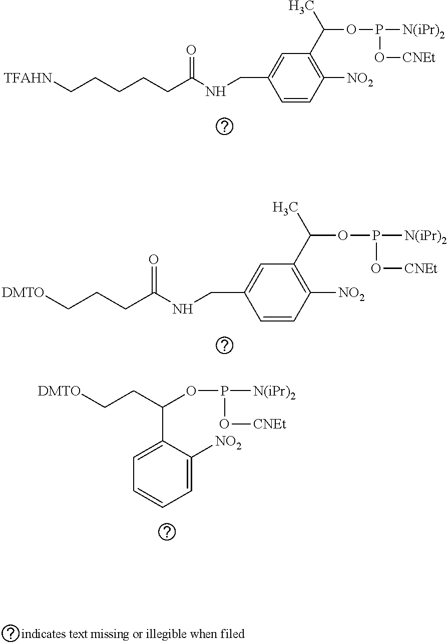

Kits

Another aspect of the present disclosure are kits, such as kits including one or more of the compounds of Formula (I). In some embodiments, the kit includes one or more compounds of Formula (I) and a polymerase. In some embodiments, the polymerase is a DNA polymerase. In some embodiments, the DNA polymerase is a thermostable DNA-dependent DAN polymerase. The kit may further include amplification primers. In some embodiments, the kit further comprises at least one of a forward primer and/or a reverse primer. In some embodiments, the kit includes a forward primer capable of hybridizing to a copy of the first oligonucleotide and a reverse primer capable of hybridizing to the second oligonucleotide. In other embodiments, the kit includes a forward primer capable of hybridizing to the first oligonucleotide and a reverse primer capable of hybridizing to a copy of the second oligonucleotide.

In other embodiments, a kit may include one or more of the compounds of Formulas (I), (II), or (III) and one or more buffers. In some embodiments, a kit comprises one or more compounds of Formula (I) and a master mix. In some embodiments, the master mix includes two or more of an enzyme, a buffer, a cofactor (e.g. MgCl₂ or MgSO₄), water, and dNTPs. In some embodiments, the master mix further includes template DNA.

In other embodiments, a kit may include a compound of Formula (II) and a compound of Formula (III). In some embodiments, the compound of Formula (II) includes a first reactive group capable of reacting with a second reactive group of the compound of Formula (III).

In some embodiments, the first reactive group comprises an alkyne moiety; and the second reactive group comprises an azide moiety. In some embodiments, the alkyne moiety is DBCO. In some embodiments, the first reactive group comprises a maleimide moiety; and the second reactive group comprises a thiol moiety. In some embodiments, the first reactive group comprises an alkene moiety and the second reactive group comprises a tetrazine moiety. In some embodiments, both the first and second reactive groups are amino moieties, and wherein the kit further comprises s-trichlorotriazine.

In some embodiments, any of the compounds of Formulas (I), (II), and/or (III) may be included in a reaction vessel, together with one or more additional components. As used herein, the term “reaction vessel” generally refers to any container, chamber, device, or assembly, in which a reaction can occur in accordance with the present teachings. In some embodiments, the reaction vessel includes a well of a dPCR chip. In some embodiments, dPCR chips may include, for example, a silicon substrate etched with nano-scale or smaller reaction wells. In some embodiments, a dPCR chip has a low thermal mass. For example, the chip may be constructed of thin, highly conductive materials that do not store heat energy. In some embodiments, a dPCR chip has a surface area of from about 50 mm² to about 150 mm². In some embodiments a dPCR chip has a surface area of about 100 mm². Limiting the surface area may allow for greater uniformity of heating of the chip during melt analysis and a reduction in run-to-run variation in the melt cure analysis, a reduction in errors in melt curve generation, and increased discrimination of melt curves in the analysis. Other dPCR chips are describes in PCT Publication No. WO/2016/133783, the disclosure of which is hereby incorporated by reference herein in its entirety.

Methods

Another aspect of the present disclosure is a method of detecting one or more gene fusions where one fusion partner is unknown. In some embodiments, the methods utilize one or more of the compounds of Formula (I). In some embodiments, the method further comprises, amplifying nucleic acids and/or forming a library of amplified nucleic acids. In some embodiments, the method further comprises sequencing a library of amplified nucleic acids thereby detecting one or more genomic rearrangements in the sample. These and other steps of the method are described herein.

Gene fusions are a common occurrence in cancer. Clinical tests for gene fusions enable detection and diagnosis of cancer, tracking tumor burden over time, and developing an individualized treatment protocol for a cancer patient. Of special utility are blood-based methods of detecting gene fusions. Blood based methods access patient's cell-free nucleic acids (cfDNA and cfRNA), which includes circulating tumor nucleic acids (ctDNA and ctRNA). While blood-based tests are less invasive than a biopsy, the major difficultly is detecting very small amounts of tumor-derived nucleic acid mixed with normal, non-tumor derived nucleic acid. Several commercially available tests are able to detect mutations in ctDNA including single nucleotide variations (SNVs), copy number variations (CNVs) and gene fusions (e.g., AVENIO ctDNA Test Kit, Roche Sequencing Solutions, Pleasanton, Cal.)

For some cancer-related gene fusions, detecting fusion products in ctDNA is further complicated by occurrence of multiple fusion partners. Tumor related genes with promiscuous fusions include many examples such as NTRK 1, 2 and 3, and FGFR 2 and 3.

Sample

The methods of the present disclosures utilizes a sample containing one or more nucleic acids, including one or more target nucleic acids. In some embodiments, the sample is derived from a subject or a patient. In some embodiments the sample may comprise a fragment of a solid tissue or a solid tumor derived from the subject or the patient, e.g., by biopsy. The sample may also comprise body fluids (e.g., urine, sputum, serum, plasma or lymph, saliva, sputum, sweat, tear, cerebrospinal fluid, amniotic fluid, synovial fluid, pericardial fluid, peritoneal fluid, pleural fluid, cystic fluid, bile, gastric fluid, intestinal fluid, or fecal samples). The sample may comprise whole blood or blood fractions where normal or tumor cells may be present. In some embodiments, the sample, especially a liquid sample may comprise cell-free material such as cell-free DNA or RNA including cell-free tumor DNA or tumor RNA of cell-free fetal DNA or fetal RNA. In some embodiments, the sample is a cell-free sample, e.g., cell-free blood-derived sample where cell-free tumor DNA or tumor RNA or cell-free fetal DNA or fetal RNA are present. In other embodiments, the sample is a cultured sample, e.g., a culture or culture supernatant containing or suspected to contain nucleic acids derived from the cells in the culture.

In some embodiments, the sample is a representative sample. In some embodiments, the representative sample is prepared from a tumor sample, a lymph node sample, a blood sample, and/or other tissue samples which are homogenized (alone or together). “Homogenization” refers to a process (such as a mechanical process and/or a biochemical process) whereby a biological sample is brought to a state such that all fractions of the sample are equal in composition. Representative samples (as defined herein) may be prepared by removal of a portion of a sample that has been homogenized. A homogenized sample (a “homogenate”) is mixed well such that removing a portion of the sample (an aliquot) does not substantially alter the overall make-up of the sample remaining and the components of the aliquot removed is substantially identical to the components of the sample remaining. In the present disclosure the “homogenization” will in general preserve the integrity of the majority of the cells within the sample, e.g., at least 50% of the cells in the sample will not be ruptured or lysed as a result of the homogenization process. In other embodiments, homogenization will preserve the integrity of at least 80% of the cells in the sample. In other embodiments, homogenization will preserve the integrity of at least 85% of the cells in the sample. In other embodiments, homogenization will preserve the integrity of at least 90% of the cells in the sample. In other embodiments, homogenization will preserve the integrity of at least 95% of the cells in the sample. In other embodiments, homogenization will preserve the integrity of at least 96 of the cells in the sample. In other embodiments, homogenization will preserve the integrity of at least 97% of the cells in the sample. In other embodiments, homogenization will preserve the integrity of at least 98% of the cells in the sample. In other embodiments, homogenization will preserve the integrity of at least 99% of the cells in the sample. In other embodiments, homogenization will preserve the integrity of at least 99.9% of cells in the same. The homogenates may be substantially dissociated into individual cells (or clusters of cells) and the resultant homogenate or homogenates are substantially homogeneous (consisting of or composed of similar elements or uniform throughout).

In some embodiments, the input sample comprises a representative sample of cells derived from a tumor sample, lymph node sample, blood sample, or any combination thereof. In some embodiments, the input sample is derived from a human patient or mammalian subject (i) diagnosed with cancer, (ii) suspected of having cancer, (iii) at risk of developing cancer; (iv) at risk of relapse or recurrence of cancer; and/or (v) suspected of having cancer recurrence. In other embodiments, the input sample is derived from a healthy human patient or mammalian subject. Additional methods of generating representative samples and/or preparing representative samples for downstream processing are described in PCT Application No. PCT/US19/62857, the disclosure of which is hereby incorporated by reference herein in its entirety.

Target Nucleic Acids

Target nucleic acids are the nucleic acid of interest that may be present in the sample. Each target is characterized by its nucleic acid sequence. The present disclosure enables detection of one or more RNA or DNA targets. In some embodiments, the DNA target nucleic acid is a gene or a gene fragment (including exons and introns) involved in a fusion event or an intergenic region where a fusion breakpoint is located. The RNA target nucleic acid is a transcript or a portion of the transcript of a gene or coding sequence resulting from fusion. In some embodiments, the target nucleic acid comprises a biomarker, i.e., a gene whose variants such as gene fusion are associated with a disease or condition. For example, the target nucleic acids can be selected from panels of disease-relevant markers described in U.S. patent application Ser. No. 14/774,518 filed on Sep. 10, 2015. Such panels are available as AVENIO ctDNA Analysis kits (Roche Sequencing Solutions, Pleasanton, Cal.)

Of special interest are target genes known to undergo gene fusions in tumors. For example, ALK, RET, ROS, FGFR^{2,} FGFR³ and NTRK1 are known to undergo fusions resulting in an abnormally active kinase phenotype. Other genes known or expected to undergo fusions relevant for cancer include ALK, PPARG, BRAF, EGFR, FGFR^{1,} FGFR^{2,} FGFR^{3,} MET, NRG1, NTRK1, NTRK2, NTRK3, RET, ROS1, AXL, PDGFRA, PDGFB , ABL1, ABL2, AKT1, AKT2, AKT3, ARHGAP26, BRD3, BRD4, CRLF2, CSF1R, EPOR, ERBB2, ERBB4, ERG, ESR1, ESRRA, ETV1, ETV4, ETVS, ETV6, EWSR1, FGR, IL2RB, INSR, JAK1, JAK2, JAK3, KIT, MAML2, MAST1, MAST2, MSMB, MUSK, MYB, MYC, NOTCH1, NOTCH2, NUMBL, NUT, PDGFRB, PIK3CA, PKN1, PRKCA, PRKCB, PTK2B, RAF1, RARA, RELA, RSPO2, RSPO3, SYK, TERT, TFE3, TFEB, THADA, TMPRSS2, TSLP, TY, BCL2, BCL6, BCR, CAMTA1, CBFB, CCNB3, CCND1, CIC, CRFL2, DUSP22, EPC1, FOXO1, FUS, GLI1, GLIS2, HMGA2, JAZF1, KMT2A, MALT1, MEAF6, MECOM, MKL1, MKL2, MTB, NCOA2, NUP214, NUP98, PAXS, PDGFB, PICALM, PLAG1, RBM15, RUNX1, RUNX1T1, SS18, STAT6, TAF15, TAL1, TCF12, TCF3, TFG, TYK2, USP6, YWHAE, AR, BRCA1, BRCA2, CDKN2A, ERB84, FLT3, KRAS, MDM4, MYBL1, NF1, NOTCH4, NUTM1, PRKACA, PRKACB, PTEN, RAD51B, and RB1.

In some embodiments, the target nucleic acid is RNA (including mRNA). In such embodiments, the DNA polymerase extending the compound of Formula (I) is a reverse transcriptase. In other embodiments, the target nucleic acid is DNA, including cellular DNA or cell-free DNA (cfDNA) including circulating tumor DNA (ctDNA) and cell-free fetal DNA. In such embodiments, the DNA polymerase extending the compound of Formula (I) is any DNA polymerase, e.g. any B family DNA polymerase. The target nucleic acid may be present in a short or long form. In some embodiments, longer target nucleic acids are fragmented by enzymatic or physical treatment as described below. In some embodiments, the target nucleic acid is naturally fragmented, e.g., includes circulating cell-free DNA (cfDNA) or chemically degraded DNA such as the one found in chemically preserved or ancient samples. In some embodiments, the ctDNA or cfDNA is derived from a representative sample (see PCT Application No. PCT/U.S. Ser. No. 19/62857, the disclosure of which is hereby incorporated by reference herein in its entirety).

DNA Isolation

In some embodiments, the method of the present disclosure comprises a step of isolating nucleic acids. Generally, any method of nucleic acid extraction that yields isolated nucleic acids comprising DNA, RNA or a mixture of DNA and RNA may be used. Genomic DNA or cellular RNA or a mixture of DNA and RNA may be extracted from tissues, cells, or liquid biopsy samples (including blood or plasma samples) using solution-based or solid-phase based nucleic acid extraction techniques. Nucleic acid extraction can include detergent-based cell lysis, denaturation of nucleoproteins, and optionally removal of contaminants. Extraction of nucleic acids from preserved samples may further include a step of deparaffinization. Solution based nucleic acid extraction methods may comprise salting out methods or organic solvent or chaotropic methods. Solid-phase nucleic extraction methods can include but are not limited to silica resin methods, anion exchange methods or magnetic glass particles and paramagnetic beads (KAPA Pure Beads, Roche Sequencing Solutions, Pleasanton, Cal.) or AMPure beads (Beckman Coulter, Brea, Cal.)

A typical extraction method involves lysis of tissue material and cells present in the sample. Nucleic acids released from the lysed cells can be bound to a solid support (beads or particles) present in solution or in a column, or membrane where the nucleic acids may undergo one or more washing steps to remove contaminants including proteins, lipids and fragments thereof from the sample. Finally, the bound nucleic acids can be released from the solid support, column or membrane and stored in an appropriate buffer until ready for further processing. Because both DNA and RNA must be isolated, no nucleases may be used, and care should be taken to inhibit any nuclease activity during the purification process.

In some embodiments, nucleic acid isolation utilizes epitachophoresis (ETP) as described in PCT/EP2019/077714 filed on Oct. 14, 2019 and PCT/EP2018/081049 filed on Nov. 13, 2018. ETP utilizes a device with a circular arrangement of electrodes where the nucleic acid migrates and concentrates between a leading electrolyte and a trailing electrolyte. The circular configuration allows concentrating nucleic acids in a very small volume collected in the center of the device. The use of ETP is especially advantageous for blood plasma samples containing small amounts of cell-free nucleic acid in a large volume.

In some embodiments, the input DNA or input RNA require fragmentation. In such embodiments, RNA may be fragmented by a combination of heat and metal ions, e.g., magnesium. In some embodiments, the sample is heated to 85°-94° C. for 1-6 minutes in the presence of magnesium. (KAPA RNA HyperPrep Kit, KAPA Biosystems, Wilmington, Mass). DNA can be fragmented by physical means, e.g., sonication, using commercially available instruments (Covaris, Woburn. Mass.) or enzymatic means (KAPA Fragmentase Kit, KAPA Biosystems).

In some embodiments, the DNA repair enzymes target damaged bases in the isolated nucleic acids. In some embodiments, sample nucleic acid is partially damaged DNA from preserved samples, e.g., formalin-fixed paraffin embedded (FFPET) samples. Deamination and oxidation of bases can result in an erroneous base read during the sequencing process. In some embodiments, the damaged DNA is treated with uracil N-DNA glycosylase (UNG/UDG) and/or 8-oxoguanine DNA glycosylase.

The methods of the present disclosure are applicable to multiple different types of nucleic acids. In some embodiments, the methods of the present disclosure utilizes isolated DNA (i.e., DNA separated from RNA by RNase digestion). In some embodiments, the methods of the present disclosure utilizes isolated RNA (i.e., RNA separated from DNA by DNase digestion). In yet other embodiments, the methods of the present disclosure utilizes a mixture of DNA and RNA (i.e., isolated nucleic acids not treated with a nuclease).

Enrichment

In some embodiments, the methods of the present disclosure further comprises a step of target enrichment. In some embodiments, the method utilizes a pool of oligonucleotide probes (e.g., capture probes). In some embodiments, enrichment is by subtraction in which case capture probes are capable of hybridizing to abundant undesired sequences including ribosomal RNA (rRNA) or abundantly expressed genes (e.g., globin). In the case of subtraction, the undesired sequences are captured by the capture probes, removed from the solution of target nucleic acids and discarded. Removal may be accomplished by utilizing capture probes with a binding moiety that can be captured on solid support. In other embodiments, enrichment is by retention in which case, capture probes are capable of hybridizing to one or more target sequences, i.e., known sequences of the fusion partner genes. In some embodiments, the target sequences are hybridized to gene-specific capture probes and removed from the solution, e.g., utilizing capture probes with a binding moiety that can be captured on solid support. The captured target-probe hybrids are retained while the remainder of the solution containing non-target sequences is discarded.

For enrichment, the capture probes may be free in solution or fixed to solid support. The probes may also comprise a binding moiety (e.g., biotin) and be capable of being captured on solid support (e.g., avidin or streptavidin containing support material).

Contacting the Sample or Target Enriched Sample with a Linked Primer, Such as with a Compound of Formula (I)

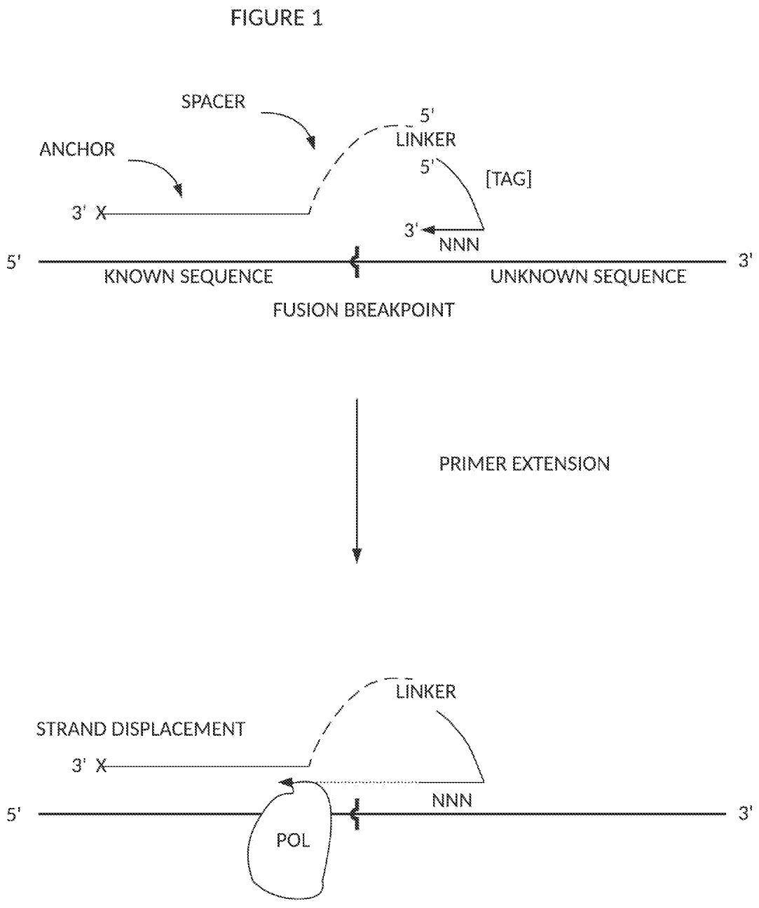

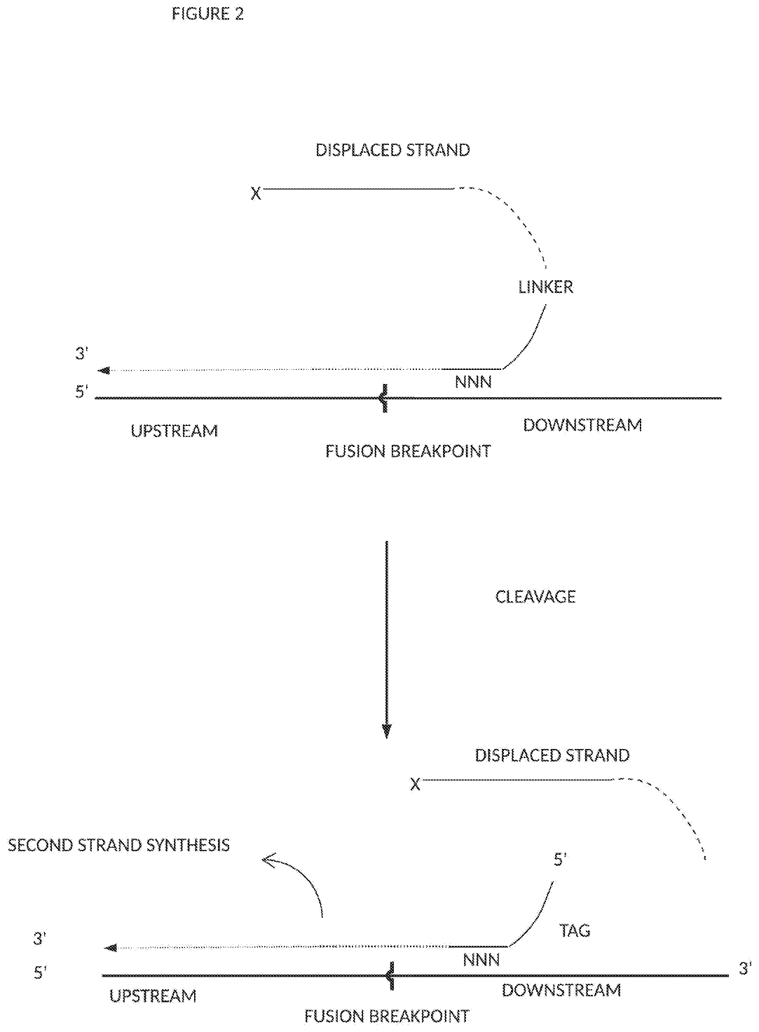

Referring to FIG. 1 (bottom) and FIG. 2, the present disclosure provides a method of detecting a gene fusion by contacting a sample with a linked primer (such as any of those of Formula (I). In some embodiments, the linked primer comprise a first oligonucleotide sequence (e.g. “Olig1” of Formula (I)) coupled directly or indirectly through a linkage (e.g. group “Z” of Formula (I)) to a second oligonucleotide sequence (e.g. “Olig2” of Formula (I)). In some embodiments, and as depicted in FIG. 1, the linked primer comprises a first oligonucleotide sequence (left side, “Olig1” of Formula (I)) which includes an anchor sequence capable of hybridizing to a known 5′-fusion partner. The linked primer also includes a “Spacer” (e.g. the group “—([R¹]ₒ—[R²]ₚ)_{q}—” of Formula (I)). The second oligonucleotide (right side, “Olig2” of Formula (I)) comprises a random sequence (“NNN”) and an extendable 3′-end.

As shown in FIG. 1 (bottom), the sample is contacted with a nucleic acid polymerase having a polymerase activity and a strand displacement activity (“POL”). In some embodiments, the nucleic acid in the sample is DNA and a DNA-dependent DNA polymerase is used, e.g., any B-family polymerase with a strand displacement activity. In some embodiments, the nucleic acid in the sample is RNA and a reverse transcriptase is used.

In some embodiments, the nucleic acid in the sample is a mixture of DNA and RNA. Such a sample can be processed to target DNA and RNA in a single tube using the method described in U.S. Provisional Application Ser. No. 62/888963 “Single tube preparation of DNA and RNA for sequencing,” filed on August 19, 2019 and incorporated herein by reference. Briefly, the method described comprises forming cDNA with a first primer having a tag identifying the RNA starting material under conditions where DNA starting material is not reactive. After cDNA is formed, target cDNA is amplified and detected along with the target DNA by a common set of amplification primers not including the first primer. Final products originating from RNA are distinguished from final products originating from DNA by the presence of the RNA—specific tag

(“RNA-identifying tag”) introduced by the first primer. In some embodiments, the 5′-portion of the second oligonucleotide (e.g. “Olig2” of Formula (I)) includes an RNA-identifying tag.

In some embodiments, the polymerase extends the 3′-end of the second oligonucleotide (e.g. “Olig2” of Formula (I)) while displacing the anchor sequence of the first oligonucleotide (e.g. “Olig1” of Formula (I)) hybridized to the known sequence of the known gene fusion partner. (FIG. 1, bottom). In some embodiments, the extension product, referred to as a first copy strand, contains a copy of a portion of the 3′-fusion partner and a portion of the 5′-fusion partner, thereby forming a first strand copy of the gene fusion.

In some embodiments, the first copy strand is copied to form a second copy thereby forming a double-stranded copy of the gene fusion. In some embodiments, a primer complementary to a sequence in a known fusion partner can be used to form the second copy strand. In some embodiments, this primer is also an amplification primer. In some embodiments, this primer comprises one or more additional features in the 5′-portion selected from a sample barcode, a molecular barcode, a universal primer binding site, and a sequencing platform-specific primer binding site.

In some embodiments, it is desirable to remove the first oligonucleotide (e.g. “Olig1” of Formula (I)) from the first copy strand. In some embodiments, a group (e.g. the group “W” of Formula (I)) between the first and second oligonucleotides (e.g. “Olig1” and “Olig2” of Formula (I) includes a cleavable moiety. In some embodiments, the cleavable linker is selected from a photocleavable, enzymatically cleavable, chemically cleavable, or pH-sensitive group. In those embodiments including a photocleavable moiety, the photocleavable moiety may be cleaved by introducing radiation having a specific wavelength (e.g. radiation having a wavelength ranging from between about 400 nm to about 800 nm). In those embodiments including a enzymatically cleavable group, the enzymatically cleavable group may be cleaved by one of a USER enzyme, uracil-N-glycosylase, an RNase A, a beta-glucuronidase, a beta-galactosidase, or a TEV-protease. In those embodiments including a chemically cleavable group, the chemically cleavable group may be cleaved by introducing an appropriate electrophile and/or nucleophile.

In some embodiments, the compound of Formula (I) does not include a group “W” (where v=0) and a cleavable moiety is included within “Olig2.” In some embodiments, “Olig2” comprises a cleavage site comprised of one or more uracil-containing nucleotides. In some embodiments, the strand comprising the uracil-containing nucleotide (e.g., the first copy strand) is cleaved by contacting the reaction mixture with Uracil-N-DNA glycosylase (UNG), optionally in the presence of primary amines as described in U. S. Pat. No. 8,669,061. UNGs recognize uracils present in single-stranded or double-stranded DNA and cleave the N-glycosidic bond between the uracil base and the deoxyribose, leaving an abasic site. See e.g. U.S. Pat. No. 6,713,294, the disclosure of which is hereby incorporated by reference herein in its entirety.

In some embodiments, cleavage is performed by a combination of a glycosylase and an endonuclease, e.g., a mixture of Uracil DNA glycosylase (UDG) and a DNA glycosylase-lyase Endonuclease VIII. Cleaving the cleavage sites separates the first copy strand from the first oligonucleotide (e.g. “Olig1” of Formula (I)) and from the linker structure (FIG. 2, bottom). In some embodiments, the cleavage takes place prior to forming the second copy strand.

In some embodiments, the first copy strand or the double-stranded copy of the gene fusion are sequenced. In some embodiments, prior to sequencing, the first copy strand or the double-stranded copy of the gene fusion are amplified prior to sequencing. As described herein, amplification can include gene specific primers, specific primers or universal primers. Universal primer binding sites may be introduced in the 5-portions of the second oligonucleotide (e.g. “Olig2” of Formula (I)) of the linked primer or the primer used to form the second copy strand.

In some embodiments, the method is multiplexed, meaning that the method is targeting multiple genes known to be involved in gene fusion events. In such embodiments, a reaction mixture is provided which comprises two or more of the compounds of Formula (I), where each of the two or more compounds of Formula (I) have an anchor sequence specific to a particular gene known to be a involved in gene fusion. For example, the same reaction mixture may contain two or more compounds of Formula (I) with anchor sequences targeting one or more of ALK, PPARG, BRAF, EGFR, FGFR^{1,} FGFR^{2,} FGFR^{3,} MET, NRG1, NTRK1, NTRK2, NTRK3, RET, ROS1, AXL, PDGFRA, PDGFB , ABL1, ABL2, AKT1, AKT2, AKT3, ARHGAP26, BRD3, BRD4, CRLF2, CSF1R, EPOR, ERBB2, ERBB4, ERG, ESR1, ESRRA, ETV1, ETV4, ETVS, ETV6, EWSR1, FGR, IL2RB, INSR, JAK1, JAK2, JAK3, KIT, MAML2, MAST1, MAST2, MSMB, MUSK, MYB, MYC, NOTCH1, NOTCH2, NUMBL, NUT, PDGFRB, PIK3CA, PKN1, PRKCA, PRKCB, PTK2B, RAF1, RARA, RELA, RSPO2, RSPO3, SYK, TERT, TFE3, TFEB, THADA, TMPRSS2, TSLP, TY, BCL2, BCL6, BCR, CAMTA1, CBFB, CCNB3, CCND1, CIC, CRFL2, DUSP22, EPC1, FOXO1, FUS, GLI1, GLIS2, HMGA2, JAZF1, KMT2A, MALT1, MEAF6, MECOM, MKL1, MKL2, MTB, NCOA2, NUP214, NUP98, PAXS, PDGFB, PICALM, PLAG1, RBM15, RUNX1, RUNX1T1, SS18, STAT6, TAF15, TAL1, TCF12, TCF3, TFG, TYK2, USP6, YWHAE, AR, BRCA1, BRCA2, CDKN2A, ERB84, FLT3, KRAS, MDM4, MYBL1, NF1, NOTCH4, NUTM1, PRKACA, PRKACB, PTEN, RAD51B, and RB1.

In some embodiments, the linked primers are designed to accommodate short input nucleic acids. For example, cell-free DNA, including circulating tumor DNA (ctDNA) averages 175 bp in length. In such embodiments, the length of the linked primer may not exceed 175 bases.

Amplification

In some embodiments, the present disclosure comprises an amplification step. The copy strand formed as illustrated in FIG. 2 (bottom), can be copied and amplified by linear or exponential amplification. Amplification may be isothermal or involve thermocycling. In some embodiments, the amplification is exponential and involves PCR. In some embodiments, at least one gene-specific primer, e.g., a primer capable of hybridizing the known fusion partner is used for amplification. In some embodiments, the 5′-portion of the linked primer comprises a primer binding site for a second primer used in amplification. In other embodiments, universal primer binding sites are added to the nucleic acid to be amplified. In some embodiments, the universal primer binding sites may be added by ligating an adaptor comprising the universal primer binding sites. In other embodiments, the universal primer binding sites are added by extending a gene specific primer having a 5′-tail comprising the universal primer binding site. All nucleic acids having the same universal primer binding sites can be conveniently amplified with the same set of primers and under the same conditions. The number of amplification cycles where universal primers are used can be low but also can be about 10, about 20 or as high as about 30 or more cycles, depending on the amount of product needed for the subsequent steps. Because PCR with universal primers has reduced sequence bias, the number of amplification cycles need not be limited to avoid amplification bias.

Primers

In some embodiments, the present disclosure involves an amplification step utilizing a forward and a reverse primer. One or both of the forward and reverse primers may be target-specific. A target specific primer comprises at least a 3′-portion that is specific for (i.e., at least partially complementary to and forms a stable hybrid with) the target nucleic acid. If additional sequences are present, such as a barcode or a universal primer binding site, they are typically located in the 5′-portion of the primer.

In some embodiments, to amplify the copy strand formed as shown in FIG. 2 (bottom), a first primer specific for a known gene sequence upstream of the fusion breakpoint may be used. In some embodiments, a second primer is specific for the tag sequence or any other engineered sequence present in the second linked oligonucleotide.

In some embodiments, the first and the second specific primers comprise a universal primer binding site in the 5′-portion of the primer. After one or more rounds of specific amplification, universal amplification is performed.

Library

In some embodiments, the present disclosure is a library of nucleic acids enriched for fusion-specific nucleic acids as described herein. The library comprises double-stranded nucleic acid molecules flanked by adaptor sequences attached thereto as described below. The nucleic acids in the library may comprise elements such as barcodes and universal primer binding sites present in adaptor sequences as described herein below. In some embodiments, the additional elements are present in adaptors and are added to the library nucleic acids via adaptor ligation. In other embodiments, some or all of the additional elements are present in amplification primers and are added to the library nucleic acids prior to adaptor ligation by extension of the primers.

In some embodiments, the library is formed from all nucleic acids in the sample prior to the use of fusion detection linked primers described herein. In this embodiment, adaptor molecules are added to all nucleic acids in the sample. The method of detecting fusions with linked primers uses the library molecules as starting material. In some embodiments, universal amplification (with universal primers hybridizing to primer binding sites located in adaptors) takes place prior to fusion-specific amplification with linked primers. The universal amplification increases the amount of starting material for fusion-specific amplification with linked primers performed as described herein.

In some embodiments, library molecules include adaptors comprising unique molecular barcodes. Sequencing the library comprises determining the sequence of barcoded library nucleic acids, grouping the sequences into families by unique molecular barcodes, and determining a consensus read for each family thereby detecting the gene fusion.

Adaptor

In some embodiments, the present disclosure utilizes an adaptor nucleic acid. The adaptor may be added to the nucleic acid by a blunt-end ligation or a cohesive end ligation. In some embodiments, the adaptor may be added by single-strand ligation method. In some embodiments, the adaptor is added by amplification with tiled primers having the adaptor sequence in the 5′-portion of the primer. The methods and compositions useful for adding adaptors by ligation or amplification are described e.g., in U.S. Pat. Nos. 9,476,095, 9,260,753, 8,822,150, 8,563,478, 7,741,463, 8,182,989 and 8,053,192, the disclosures of which are hereby incorporated by reference herein in their entireties.

In some embodiments, adaptor molecules are in vitro synthesized artificial sequences. In other embodiments, adaptor molecules are in vitro synthesized naturally occurring sequences. In yet other embodiments, adaptor molecules are isolated naturally occurring molecules or isolated non-naturally occurring molecules.

In the case of adaptor added by ligation, the adaptor oligonucleotide can have overhangs or blunt ends on the terminus to be ligated to the target nucleic acid. In some embodiments, the adaptor comprises blunt ends to which a blunt-end ligation of the target nucleic acid can be applied. The target nucleic acids may be blunt-ended or may be rendered blunt-ended by enzymatic treatment (e.g., “end repair”). In other embodiments, the blunt-ended DNA undergoes A-tailing where a single A nucleotide is added to the 3′-end of one or both blunt ends. The adaptors described herein are made to have a single T nucleotide extending from the blunt end to facilitate ligation between the nucleic acid and the adaptor. Commercially available kits for performing adaptor ligation include AVENIO ctDNA Library Prep Kit or KAPA HyperPrep and HyperPlus kits (Roche Sequencing Solutions, Pleasanton, Cal.). In some embodiments, the adaptor ligated DNA may be separated from excess adaptors and unligated DNA.

The adaptor may further comprise features such as universal primer binding site (including a sequencing primer-binding site) a barcode sequence (including a sample barcode (SID) or a unique molecular barcode or identifier (UID or UMI). In some embodiments, the adaptors comprise all of the above features while in other embodiments, some of the features are added after adaptor ligation by extending tailed primers that contain some of the elements described above.

The adaptor may further comprise a capture moiety. The capture moiety may be any moiety capable of specifically interacting with another capture molecule. Capture moieties-capture molecule pairs include avidin (streptavidin)-biotin, antigen-antibody, magnetic (paramagnetic) particle-magnet, or oligonucleotide-complementary oligonucleotide. The capture molecule can be bound to a solid support so that any nucleic acid on which the capture moiety is present is captured on solid support and separated from the rest of the sample or reaction mixture. In some embodiments, the capture molecule comprises a capture moiety for a secondary capture molecule. For example, a capture moiety in the adaptor may be a nucleic acid sequence complementary to a capture oligonucleotide. The capture oligonucleotide may be biotinylated so that adapted nucleic acid-capture oligonucleotide hybrid can be captured on a streptavidin bead.

In some embodiments, the adaptor-ligated nucleic acid is enriched via capturing the capture moiety and separating the adaptor-ligated target nucleic acids from unligated nucleic acids in the sample.

In some embodiments, the stem portion of the adaptor includes a modified nucleotide increasing the melting temperature of the capture oligonucleotide, e.g., 5-methyl cytosine, 2,6-diaminopurine, 5-hydroxybutyn-2′-deoxyuridine, 8-aza-7-deazaguanosine, a ribonucleotide, a 2′O-methyl ribonucleotide or a locked nucleic acid. In another aspect, the capture oligonucleotide is modified to inhibit digestion by a nuclease, e.g., by a phosphorothioate nucleotide.

In some embodiments, adaptor sequences are added to the copy strand formed as shown in FIG. 2 (bottom) either by ligation of adaptors or by amplification with tailed primers. The adaptors may be added to either a single strand or a double-stranded molecule comprising the copy strand shown in FIG. 2.

Barcodes

In some embodiments, the present disclosure utilizes a barcode. Detecting individual molecules typically requires molecular barcodes such as described in U.S. Pat. Nos. 7,393,665, 8,168,385, 8,481,292, 8,685,678, and 8,722,368. A unique molecular barcode is a short artificial sequence added to each molecule in the patient's sample typically during the earliest steps of in vitro manipulations. The barcode marks the molecule and its progeny. The unique molecular barcode (UID) has multiple uses. Barcodes allow tracking each individual nucleic acid molecule in the sample to assess, e.g., the presence and amount of circulating tumor DNA (ctDNA) molecules in a patient's blood in order to detect and monitor cancer without a biopsy (Newman, A., et al., (2014) An ultrasensitive method for quantitating circulating tumor DNA with broad patient coverage, Nature Medicine doi:10.1038/nm.3519).

A barcode can be a multiplex sample ID (MID) used to identify the source of the sample where samples are mixed (multiplexed). The barcode may also serve as a unique molecular ID (UID) used to identify each original molecule and its progeny. The barcode may also be a combination of a UID and an MID. In some embodiments, a single barcode is used as both UID and MID. In some embodiments, each barcode comprises a predefined sequence. In other embodiments, the barcode comprises a random sequence. In some embodiments of the present disclosure, the barcodes are between about 4-20 bases long so that between 96 and 384 different adaptors, each with a different pair of identical barcodes are added to a human genomic sample. A person of ordinary skill would recognize that the number of barcodes depends on the complexity of the sample (i.e., expected number of unique target molecules) and would be able to create a suitable number of barcodes for each experiment.

Unique molecular barcodes can also be used for molecular counting and sequencing error correction. The entire progeny of a single target molecule is marked with the same barcode and forms a barcoded family. A variation in the sequence not shared by all members of the barcoded family is discarded as an artifact and not a true mutation. Barcodes can also be used for positional deduplication and target quantification, as the entire family represents a single molecule in the original sample (Newman, A., et al., (2016) Integrated digital error suppression for improved detection of circulating tumor DNA, Nature Biotechnology 34:547).

In some embodiments, the number of UIDs in the plurality of adaptors or barcode-containing primers may exceed the number of nucleic acids in the plurality of nucleic acids. In some embodiments, the number of nucleic acids in the plurality of nucleic acids exceeds the number of UIDs in the plurality of adaptors.

Purification

In some embodiments, the present disclosure comprises intermediate purification steps. For example, any unused oligonucleotides such as excess primers and excess adaptors are removed, e.g., by a size selection method selected from gel electrophoresis, affinity chromatography and size exclusion chromatography. In some embodiments, size selection can be performed using Solid Phase Reversible Immobilization (SPRI) technology from Beckman Coulter (Brea, Cal.). In some embodiments, a capture moiety is used to capture and separate adaptor-ligated nucleic acids from unligated nucleic acids or excess primers from the products of exponential amplification. In some embodiments, the excess oligonucleotides including unused primers or adaptors are removed using a specific capture nucleic acid that forms a closed circular structure that encloses the oligonucleotide to be removed as described in the U.S. Application Ser. No. 63/021875 “Removal of excess oligonucleotides from a reaction mixture,” filed on May 8, 2020.

Sequencing

In some embodiments, the copy strands, double stranded copies of gene fusion sequences and libraries of nucleic acids including gene fusion sequences, or amplicons thereof can be subjected to nucleic acid sequencing. Sequencing may be performed according to any method known to those of ordinary skill in the art. In some embodiments, sequencing methods include Sanger sequencing and dye-terminator sequencing, as well as next-generation sequencing technologies such as pyrosequencing, nanopore sequencing, micropore-based sequencing, nanoball sequencing, MPSS, SOLiD, Illumina, Ion Torrent, Starlite, SMRT, tSMS, sequencing by synthesis, sequencing by ligation, mass spectrometry sequencing, polymerase sequencing, RNA polymerase (RNAP) sequencing, microscopy-based sequencing, microfluidic Sanger sequencing, microscopy-based sequencing, RNAP sequencing, tunneling currents DNA sequencing, and in vitro virus sequencing. See WO2014144478, WO2015058093, WO2014106076 and WO2013068528, each of which is hereby incorporated by reference in its entirety.

In some embodiments, sequencing can be performed by a number of different methods, such as by employing sequencing by synthesis technology. Sequencing by synthesis according to the prior art is defined as any sequencing method which monitors the generation of side products upon incorporation of a specific deoxynucleoside-triphosphate during the sequencing reaction (Hyman, 1988, Anal. Biochem. 174:423-436; Rhonaghi et al., 1998, Science 281:363-365). One prominent embodiment of the sequencing by synthesis reaction is the pyrophosphate sequencing method. In this case, generation of pyrophosphate during nucleotide incorporation is monitored by an enzymatic cascade which results in the generation of a chemo-luminescent signal. The 454 Genome Sequencer System (Roche Applied Science cat. No. 04 760 085 001), an example of sequence by synthesis, is based on the pyrophosphate sequencing technology. For sequencing on a 454 GS20 or 454 FLX instrument, the average genomic DNA fragment size is in the range of 200 or 600 bp, respectively, as described in the product literature.

In some embodiments, a sequencing by synthesis reaction can alternatively be based on a terminator dye type of sequencing reaction. In this case, the incorporated dye deoxynucleotriphosphates (ddNTPs) building blocks comprise a detectable label, which is preferably a fluorescent label that prevents further extension of the nascent DNA strand. The label is then removed and detected upon incorporation of the ddNTP building block into the template/primer extension hybrid for example by using a DNA polymerase comprising a 3′-5′ exonuclease or proofreading activity.

In some embodiments, sequencing is performed using a next-generation sequencing method such as that provided by Illumina, Inc. (the “Illumina Sequencing Method”). Without wishing to be bound by any particular theory, the Illumina next-generation sequencing technology uses clonal amplification and sequencing by synthesis (SBS) chemistry to enable rapid, accurate sequencing. The process simultaneously identifies DNA bases while incorporating them into a nucleic acid chain. Each base emits a unique fluorescent signal as it is added to the growing strand, which is used to determine the order of the DNA sequence.

In some embodiments, the sequencing method is a high-throughput single molecule sequencing method utilizing nanopores. In some embodiments, the nucleic acids and libraries of nucleic acids formed as described herein are sequenced by a method involving threading through a biological nanopore (see U.S. Pat. No. 1,033,7060, the disclosure of which is hereby incorporated by reference herein in its entirety) or a solid-state nanopore (see U.S. Pat. No. 10,288,599, US20180038001, U.S. Pat. No. 10,364,507, the disclosures of which are hereby incorporated by reference herein in their entireties). In other embodiments, sequencing involves threading tags through a nanopore. (see U.S. Pat. No. 8,461,854, the disclosure of which is hereby incorporated by reference herein in its entirety) or any other presently existing or future DNA sequencing technology utilizing nanopores.

In other embodiments, sequencing is performed by other suitable technologies of high-throughput single molecule sequencing. include the Illumina HiSeq platform (Illumina, San Diego, Cal.), Ion Torrent platform (Life Technologies, Grand Island, NY), Pacific BioSciences platform utilizing the Single Molecule Real-Time (SMRT) technology (Pacific Biosciences, Menlo Park, Cal.) or any other presently existing or future DNA sequencing technology that does or does not involve sequencing by synthesis.

The sequencing step may utilize platform-specific sequencing primers. Binding sites for these primers may be introduced in 5′-portions of the amplification primers used in the amplification step. If no primer sites are present in the library of barcoded molecules, an additional short amplification step introducing such binding sites may be performed.

In some embodiments, the sequencing step involves sequence analysis. In some embodiments, the analysis includes a step of sequence aligning. In some embodiments, aligning is used to determine a consensus sequence from a plurality of sequences, e.g., a plurality having the same barcodes (UID). In some embodiments barcodes (UIDs) are used to determine a consensus from a plurality of sequences all having an identical barcode (UID). In other embodiments, barcodes (UIDs) are used to eliminate artifacts, i.e., variations existing in some but not all sequences having an identical barcode (UID). Such artifacts resulting from PCR errors or sequencing errors can be eliminated.

In some embodiments, the number of each sequence in the sample can be quantified by quantifying relative numbers of sequences with each barcode (UID) in the sample. Each UID represents a single molecule in the original sample and counting different UIDs associated with each sequence variant can determine the fraction of each sequence in the original sample. A person skilled in the art will be able to determine the number of sequence reads necessary to determine a consensus sequence. In some embodiments, the relevant number is reads per UID (“sequence depth”) necessary for an accurate quantitative result. In some embodiments, the desired depth is 5-50 reads per UID.

In some embodiments, the step of sequencing further includes a step of error correction by consensus determination. Sequencing by synthesis of the circular strand of the gapped circular template disclosed herein enables iterative or repeated sequencing. Multiple reads of the same nucleotide position enable sequencing error correction through establishment of a consensus call for each nucleotide or for the entire sequence or for a part of the sequence. The final sequence of a nucleic acid strand is obtained from the consensus base determinations at each position. In some embodiments, a consensus sequence of a nucleic acid is obtained from a consensus obtained by comparing the sequences of complementary strands or by comparing the consensus sequences of complementary strands. In some embodiments, the present disclosure comprises after the sequencing step, a step of sequence read alignment and a step of generating a consensus sequence. In some embodiments, consensus is a simple majority consensus described in U.S. Pat. No. 8,535,882. In other embodiments, consensus is determined by Partial Order Alignment (POA) method described in Lee et al. (2002) “Multiple sequence alignment using partial order graphs,” Bioinformatics, 18(3):452-464 and Parker and Lee (2003) “Pairwise partial order alignment as a supergraph problem—aligning alignments revealed,” J. Bioinformatics Computational Biol., 11:1-18. Based on the number of iterative reads used to determine a consensus sequence, the sequence may be largely free or substantially free of errors.

No sequencing

In some embodiments, the copy strands, double stranded copies of gene fusion sequences and libraries of nucleic acids including gene fusion sequences, or amplicons thereof are detected without sequencing. The detection may be accomplished by amplification, including by end-point polymerase chain reaction (PCR), quantitative PCR (qPCR) or digital PCR (dPCR), including digital droplet PCR (ddPCR). In some embodiments, detection of gene fusions is quantitative, such as the type of detection enabled by qPCR and dPCR. In other embodiments, detection of gene fusion is qualitative, i.e., the read-out is the presence or absence of the fusion-specific amplification product by gel electrophoresis, capillary electrophoresis, mass-spectrometry, or another method of detecting a nucleic acid of a characteristic size or characteristic molecular weight.

dPCR

In some embodiments, gene fusion-specific amplification according to the present disclosure is conducted by digital PCR (dPCR) including digital droplet PCR (ddPCR).

Digital PCR is a method of quantitative amplification of nucleic acids described e.g., in U.S. Pat. No. 9,347,095, the disclosure of which is hereby incorporated by reference herein. The process involves partitioning a sample into reaction volumes so that each volume comprises one or fewer copies of the target nucleic acid. In some embodiments, the partitioned reaction volume is an aqueous droplet.

In some embodiments, the target nucleic acid in partitions is the copy strand. In other embodiments, the target nucleic acid in partitions is the double stranded copy of the gene fusion sequence. Each partition further comprises amplification primers, i.e., a forward and a reverse primer capable of supporting exponential amplification of the target nucleic acid. In some embodiments, the forward and a reverse primer are capable of hybridizing to the known fusion sequence and to the 5′-sequence of the second oligonucleotide (FIG. 1).

Each of the digital PCR reaction volumes further comprises a detectably-labeled probe capable of hybridizing to an amplicon of the forward and reverse primers. In some embodiments, the probe is capable of hybridizing to the known fusion sequence. In some embodiments, the probe is designed to avoid binding to the wild-type non-fusion gene sequence.

The detectably labeled probe may be labeled with a combination of a fluorophore and the exponential amplification may be performed with a nucleic acid polymerase having a 5′-3′-exonuclease activity.

In some embodiments, the method of the present disclosure comprises performing an amplification reaction with the forward and reverse primers, wherein the reaction comprises a step of detecting the amplicon with the probe and determining a number of reaction volumes where the probe has been detected thereby detecting the presence of a gene fusion in the sample.

1. A method of detecting a gene fusion in a nucleic acid sample, the method comprising (a) contacting the nucleic acid sample with (i) a nucleic acid polymerase having a polymerase activity and a strand displacement activity, and (ii) a compound having Formula (I):
[Olig1]—([R¹]ₒ—[R²]ₚ)_{q}—[L¹]ₜ—[Z]—[L²]ᵤ—[W]ᵥ—[Olig2] (I),
wherein
o is 0 or 1;
p is 0 or 1;
q is 0 or 1;
t is 0, 1 or 2;
u is 0, 1 or 2;
v is 0 or 1;
R¹ is an oligonucleotide having between about 1 and about 24 nucleotides;
R² is a substituted or unsubstituted, saturated or unsaturated, linear or cyclic aliphatic group having between 2 and about 48 carbon atoms, optionally substituted with one or more heteroatoms selected from O, N, or S;
L¹ and L² are independently a substituted or unsubstituted, saturated or unsaturated, linear or cyclic aliphatic group having between 1 and about 16 carbon atoms, optionally including with one or more heteroatoms selected from O, N, or S, and optionally including one or more carbonyl groups;
Z is a moiety selected from a triazole, a dihydropyridazine, a phosphate linkage, an amide linkage, a thioether linkage, an isooxazoline, a hydrozone, an oxime ether, and a chloro-s-triazine linkage;
W is a substituted or unsubstituted, saturated or unsaturated, aliphatic or aromatic group having between 1 and about 12 carbon atoms, optionally substituted with one or more heteroatoms selected from O, N, S, provided that W includes at least one photocleavable, enzymatically cleavable, chemically cleavable, or pH sensitive group;
Olig1 is an oligonucleotide comprising between about 1 and about 30 nucleotides and comprising an anchor sequence capable of hybridizing to a known fusion partner, and wherein Olig1 has a non-extendable 3′ end; and
Olig2 is an oligonucleotide comprising between about 1 and about 30 nucleotides and comprising an extendable 3′ end; and
(b) extending the 3′-end of Olig2 with the nucleic acid polymerase, wherein an extension product comprises a copy of a portion of an unknown fusion partner, a portion of the known fusion partner, and a fusion breakpoint, thereby forming a first strand copy of the gene fusion.

2. The method of claim 1, further comprising forming a library of double-stranded copies of the gene fusions; wherein the forming of the library comprises: attaching adaptors to copies of gene fusions wherein adaptors comprise barcodes and primer binding sites.

3. The method of claim 1, further comprising amplifying the copy of the gene fusion by a method comprising:
(a) partitioning the sample comprising the copy of the gene fusion into a plurality of reaction volumes; wherein each reaction volume comprises a forward and a reverse amplification primers capable of hybridizing to the copy strand and the complement of the copy strand, and a first detectably-labeled probe;
(b) performing an amplification reaction, wherein the reaction comprises a step of detection with the probe;
(c) determining a number of reaction volumes where the probe has been detected thereby detecting the gene fusion.

4. A compound having Formula (I),
[Olig1]—([R¹]ₒ—[R²]ₚ)_{q}—[L¹]ₜ—[Z]—[L²]ᵤ—[W]ᵥ—[Olig2] (I),
wherein
o is 0 or 1;
p is 0 or 1;
q is 0 or 1;
t is 0, 1 or 2;
u is 0, 1 or 2;
v is 0 or 1;
R¹ is an oligonucleotide having between about 1 and about 24 nucleotides;
R² is a substituted or unsubstituted, saturated or unsaturated, linear or cyclic aliphatic group having between 2 and about 48 carbon atoms, optionally substituted with one or more heteroatoms selected from O, N, or S;
L¹ and L² are independently a substituted or unsubstituted, saturated or unsaturated, linear or cyclic aliphatic group having between 1 and about 16 carbon atoms, optionally including with one or more heteroatoms selected from O, N, or S, and optionally including one or more carbonyl groups;
Z is a moiety selected from a triazole, a dihydropyridazine, a phosphate linkage, an amide linkage, a thioether linkage, an isooxazoline, a hydrozone, an oxime ether, and a chloro-s-triazine linkage;
W is a substituted or unsubstituted, saturated or unsaturated, aliphatic or aromatic group having between 1 and about 12 carbon atoms, optionally substituted with one or more heteroatoms selected from O, N, S, provided that W includes at least one photocleavable, enzymatically cleavable, chemically cleavable, or pH sensitive group;
Olig1 is an oligonucleotide having between about 1 and about 30 nucleotides, and wherein Olig1 has a non-extendable 3′ end; and
Olig2 is an oligonucleotide having between about 1 and about 30 nucleotides, and wherein Olig2 has an extendable 3′ end.

5. The compound of claim 4, wherein R² comprises a substituted or unsubstituted, saturated or unsaturated, linear or cyclic aliphatic group having between 2 and about 32 carbon atoms, optionally substituted with one or more heteroatoms selected from O, N, or S, and optionally including one or more carbonyl groups.

6. The compound of claim 4, wherein R² comprises a moiety having the structure of Formula (IVA): 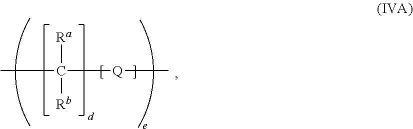
wherein d and e are integers each independently ranging from 1 to 32; Q is a bond, O, S, N(R^{c})(R^{d}) or a quaternary amine (N⁺H(R^{c})(R^{d})); R^{a} and R^{b} are independently H, a C₁-C₄ alkyl group, F, Cl, or N(^{Rc}((R^{d}); and R^{c} and R^{d} are independently CH₃ or H.

7. The compound of claim 4, wherein R² comprises a moiety having the structure of Formula (IVB): 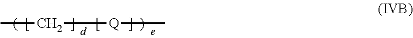
wherein d and e are integers each independently ranging from 1 to 32; Q is a bond, O, S, or N(R_{c})(R_{d}); and R_{c} and R_{d} are independently CH₃ or H.

8. The compound of claim 4, wherein at least one of L¹ or L² comprises a substituted or unsubstituted, saturated or unsaturated, linear or cyclic aliphatic group having between 1 and about 4 carbon atoms, optionally substituted with one or more heteroatoms selected from O, N, or S, and optionally including one or more carbonyl groups.

9. The compound of claim 4, wherein o+p=1, and q is 1.

10. The compound of claim 4, wherein o is 0 and p and q are both 1, R¹ comprises at least one PEG group, and L¹ comprises at least one carbonyl moiety.

11. The compound of claim 4, wherein Olig2 comprises a barcode.

12. The compound of claim 4, wherein Olig2 comprises a universal primer binding site.

13. The compound of claim 4, wherein v is 0, and Olig2 includes a cleavage site including at least one uracil-containing nucleotide.

14. The compound of claim 4, wherein Olig2 comprises a random nucleotide sequence.

15. A kit or detecting gene fusions between a known fusion partner and an unknown fusion partner, the kit comprising the compound of any one of claims 30-63, and a polymerase.

16. A kit comprising:
(a) a first compound having Formula (II):
[Olig1]—([R¹]ₒ—[R²]ₚ)_{q}—[L¹]ₜ—[X] (II),
wherein
o is 0 or 1;
p is 0 or 1;
q is 1 or 2;
t is 0, 1 or 2;
R¹ is an oligonucleotide having between 1 and about 24 nucleotides;
R² is a substituted or unsubstituted, saturated or unsaturated, linear or cyclic aliphatic group having between 2 and about 48 carbon atoms, optionally substituted with one or more heteroatoms selected from O, N, or S;
L¹ is a substituted or unsubstituted, saturated or unsaturated, linear or cyclic aliphatic group having between 1 and about 16 carbon atoms, optionally substituted with one or more heteroatoms selected from O, N, or S, and optionally including one or more carbonyl groups;
X is a dibenzocyclooctyne, a trans-cyclooctene, an alkyne, an alkene, an azide, a tetrazine, amaleimide, a N-hydroxysuccinimide, athiol, a 1,3-nitrone, an aldehyde, a ketone, a hydrazine, a hydroxylamine, an amino group, or a phosphoramidite; and
Olig1 is an oligonucleotide having between about 1 to about 30 nucleotides;
(b) a second compound having Formula (III):
[Y][L²]ᵤ—[W]ᵥ—[Olig2] (III),
wherein
u is 0, 1 or 2;
v is 0 or 1;
Y is a dibenzocyclooctyne, a trans-cyclooctene, an alkyne, an alkene, an azide, a tetrazine, amaleimide, a N-hydroxysuccinimide, athiol, a 1,3-nitrone, an aldehyde, a ketone, a hydrazine, a hydroxylamine, an amino group, or a phosphoramidite;
L² is a substituted or unsubstituted, saturated or unsaturated, linear or cyclic aliphatic group having between 1 and 16 carbon atoms, optionally including with one or more heteroatoms selected from O, N, or S, and optionally including one or more carbonyl groups;
W is a substituted or unsubstituted, saturated or unsaturated, aliphatic or aromatic group having between 1 and 12 carbon atoms, optionally substituted with one or more heteroatoms selected from O, N, S, provided that W includes a photocleavable, enzymatically cleavable, chemically cleavable, or pH sensitive group; and
Olig2 is an oligonucleotide having between about 1 and about 30 nucleotides.

17. Use of the compound of any of one of claims 4-14 or a kit of claim 15-15 in sequencing a nucleic acid molecule.
